(19) United States
(12) Patent Application Publication
(10) Pub. No.: US 2026/0201311 A1
(43) Pub. Date: Jul. 16, 2026

(54) DEVICES, SYSTEMS, AND METHODS FOR ORGANOID ANALYSIS

(71) Applicant: Diagnostic Biochips, Inc, Glen Burnie, MD (US)

(72) Inventors: Brian Glenn JAMIESON, Glen Burnie, MD (US); Fan WU, Silver Spring, MD (US); Dongyoul LEE, Laurel, MD (US); Andrew S. DIRKS, Annapolis, MD (US); Nicholas Talbot WATKINS, Severna Park, MD (US)

(73) Assignee: Diagnostic Biochips, Inc, Glen Burnie, MD (US)

(21) Appl. No.: 19/427,308

(22) Filed: Dec. 19, 2025

Related U.S. Application Data
(60) Provisional application No. 63/737,166, filed on Dec. 20, 2024.
(60) Provisional application No. 63/911,758, filed on Nov. 5, 2025.

(51) Int. Cl. C12M 1/36; C12M 1/32; G01N 27/02; G06T 7/73

(52) U.S. Cl. CPC C12M 41/48; C12M 23/12; G01N 27/028; G06T 7/73; G06T 2207/10068; G06T 2207/30072; G06T 2207/30204

(57) ABSTRACT

An organoid probing system includes a probe head having a probe, a first stage along which the probe head is movable in an X direction, and a second stage configured to move a microwell plate in a Y direction. The microwell plate has a microwell containing an organoid. The probe head is movable in a Z direction to insert the probe into the microwell or retract the probe away from the organoid in order to measure a property of the organoid.

### CROSS REFERENCE TO RELATED APPLICATIONS

This application claims the benefit of priority to U.S. Provisional Application No. 63/737,166, filed Dec. 20, 2024 and U.S. Provisional Application No. 63/911,758, filed Nov. 5, 2025, and incorporated by reference herein in their entireties.

### TECHNICAL FIELD

Various aspects of this disclosure relate generally to devices, system, and methods for organoid analysis. In particular, aspects of this disclosure relate to automated systems for probing organoids, plates having wells for containing organoids, and systems with integrated organoid probes.

### BACKGROUND

Organoids are three-dimensional, differentiated cell structures that are cultured from induced pluripotent stem cells (iPSCs) and can be used as models of specific organs such as the brain or heart. Organoids are utilized to, for example, develop and discover drugs or other therapies and/or medical techniques. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

Each of the aspects disclosed herein may include one or more aspects of the features described in connection with any of the other disclosed aspects.

According to an aspect of the present disclosure, an organoid probing system is provided. The system includes a probe head having a probe. The system includes a first stage along which the probe head is movable in an X direction. The system includes a second stage configured to move a microwell plate in a Y direction. The microwell plate has a microwell containing an organoid. The probe head is movable in a Z direction to insert the probe into the microwell or retract the probe away from the organoid in order to measure a property of the organoid.

According to other aspects of the present disclosure, the organoid probing system may include one or more of the following features. The microwell plate may have a plurality of microwells each containing an organoid. The system may further include a washing station containing a fluid for washing the probe. The system may further include a camera. The microwell plate may include at least one fiducial, and the camera may be configured to use the at least one fiducial to align the probe head relative to the microwell. The system may further include a track coupled to the first stage and a slot defined within the track. The probe head may be movable along the track through the slot. The system may further include a controller configured to control movement of the first stage and the second stage based on closed-loop feedback from electrophysiology data gathered from the probe. The system may further include at least one rotational stage to provide additional degrees of freedom for probe positioning. The system may further include an enclosure that provides environmental control and acts as a faraday cage to shield the probe head from electromagnetic interference. The probe head may include a motherboard and a probe board. The motherboard and the probe board may be coupled to one another approximately orthogonally. A cover may cover at least a portion of the probe in at least some configurations. The probe head may include a plurality of probes that are arranged linearly, such that a line extends through all of the probes. The microwell plate may include a plurality of microwells. The probe head may include a cover that at least partially covers the probe. The cover may have a shape corresponding to a shape of a negative space between adjacent microwells of the microwell plate. The microwell may include a guide.

According to another aspect of the present disclosure, a microwell plate for organoids is provided. The microwell plate includes a plurality of wells. Each well has a first portion, a second portion, and a third portion. The first portion has a different shape than the second portion. The second portion has a different shape than the third portion.

According to other aspects of the present disclosure, an organoid probing system may include the microwell plate and may further include a probe head having a probe and a stage along which the probe head is movable. The probe head may be configured to insert a probe of the probe head into a microwell of the plurality of microwells in order to measure a property of an organoid within the microwell. The probe may be configured to extend at least into the second portion of the microwell. At least the third portion may include a surface feature. The surface feature may increase fixation of an organoid within a well of the plurality of wells or may reduce contact between an organoid within a well of the plurality of wells and a surface of the well.

According to another aspect of the present disclosure, an organoid probing system is provided. The system includes a probe head. The probe head includes a motherboard. The probe head includes a probe board coupled to the motherboard and having a probe extending therefrom. The probe board is configured as a consumable component removable from the motherboard. The system includes a stage along which the probe head is movable. The system includes a microwell plate having a microwell containing an organoid. The probe head is configured to insert the probe into the microwell in order to measure a property of the organoid.

According to other aspects of the present disclosure, the organoid probing system may further include a first stage along which the probe head is movable in an X direction and a second stage configured to move a microwell plate in a Y direction. The probe head may be movable in a Z direction to insert the probe into the microwell or retract the probe away from the organoid in order to measure a property of the organoid.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated in and constitute a part of this application, illustrate exemplary aspects of this disclosure and, together with the description, serve to explain the principles of this disclosure.

FIG. 1 illustrates an organoid probing system.

FIGS. 2A and 2B illustrate a stage system of the organoid probing system.

FIGS. 3A and 3B illustrate features of a probe head.

FIGS. 4A and 4B illustrate internal features of the probe head.

FIGS. 5A and 5B illustrate components of the probe head.

FIG. 6 illustrates an alternative probe head.

FIG. 7 illustrates an alternative organoid probing system.

FIG. 8 illustrates a stage system of the organoid probing system.

FIGS. 9, 10, and 11 illustrate another organoid probing system.

FIG. 12 illustrates various housing and door configurations.

FIGS. 13A-13D illustrate an alternative probe head.

FIGS. 14A-14C illustrate another probing system.

FIGS. 15A-15E illustrate another probing system.

FIGS. 16A and 16B illustrate another probing system.

FIG. 17 illustrates another probing system.

FIGS. 18A-18C illustrate features of a microwell plate.

FIGS. 18D-18F illustrate alternative well configurations.

FIGS. 19 and 20 illustrate alternative well geometries.

FIGS. 21 and 22 illustrate additional well geometry variations.

FIGS. 23A-23C illustrate a modular microwell plate system.

FIGS. 24A and 24B illustrate an alternative well.

FIG. 25 illustrates alternative well configurations.

FIGS. 26A, 26B, and 27 illustrate alternative well configurations.

FIG. 28 illustrates another well configuration.

FIGS. 29 and 30 illustrate further well configurations.

FIG. 31 illustrates interaction between a probe head and a well.

FIGS. 32A and 32B illustrate another organoid probing system.

FIG. 33A-33C illustrate another organoid probing system.

FIG. 34 illustrates a well of the probing system of FIGS. 33A-33C

FIGS. 35A-35C illustrate a microwell plate.

FIG. 36 illustrates a microwell plate.

FIGS. 37A-37C illustrate another organoid probing system.

FIGS. 38A-38C illustrate a probe head.

FIG. 39 illustrates an organoid plate system

FIG. 40 illustrates data processing from probe measurements.

FIG. 41 illustrates a training process for a machine learning model.

FIGS. 42A-42B illustrate a microwell plate.

FIGS. 43A-43C illustrate a microwell plate.

FIGS. 44A-44C illustrate a microwell plate.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Particular aspects of the disclosure are described in greater detail below. The terms and definitions provided herein control, if in conflict with terms or definitions incorporated by reference. Wherever possible, the same or similar reference numbers will be used through the drawings to refer to the same or like parts. Where feasible, reference numbers ending in the same tens and ones digits refer to corresponding components.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “or” is used disjunctively, such that “at least one of A or B” includes, (A), (B), (A and A), (A and B), etc. The term “or” includes “and/or.” Relative terms such as “about,” “substantially,” and “approximately,” etc., are used to indicate a possible variation of ±10% of the stated numeric value or range. The term “diameter” includes a width of a non-circular element, unless otherwise specified. The term “circumference” or “circumferential” refers to the perimeter of a non-circular element, unless otherwise specified.

The following description sets forth exemplary aspects of this disclosure. It should be recognized, however, that such description is not intended as a limitation on the scope of this disclosure. Rather, the description also encompasses combinations and modifications to those exemplary aspects described herein.

Various aspects of this disclosure relate to automated organoid probing systems that provide enhanced measurement capabilities for electrophysiological analysis of three-dimensional cell structures. An organoid probing system may include a probe head having a probe, a stage along which probe head is movable, and a microwell plate having a microwell containing an organoid. Probe head may be configured to insert a probe into the microwell to measure properties of organoid, enabling systematic analysis of organoid electrical activity patterns. The automated positioning capabilities provided by the stage system may enable precise probe placement while maintaining measurement consistency across multiple organoid samples.

Microwell plates disclosed herein may include specialized well geometries that optimize organoid positioning and containment during measurement operations. A microwell plate for organoids may include wells having a first portion, a second portion, and a third portion, wherein the first portion has a different shape than the second portion, and wherein the second portion has a different shape than the third portion. The progressive shape changes may provide systematic guidance that directs organoids toward optimal positioning within wells while providing increasing containment as organoids descend into wells. Surface features may be incorporated to enhance organoid fixation or reduce contact between organoids and well surfaces, optimizing both positioning stability and organoid health during measurement operations.

Multi-well organoid probing capabilities may be achieved through integrated probe assembly configurations that enable simultaneous measurement operations across multiple organoids. An organoid probing system may include a microwell plate having multiple microwells, each configured to contain an organoid, and a probe assembly having multiple drivers, wherein each driver is configured to drive a probe into a respective microwell. The probe assembly may form a lid for the microwell plate, creating an integrated measurement system that combines probe positioning capabilities with microwell containment functions while maintaining independent control over individual probe insertion parameters.

Organoid retention systems may provide enhanced positioning stability through mechanical engagement features that secure organoids during probe insertion operations. A system for containing organoids may include a microwell plate having a retention feature protruding from a bottom of a microwell, and a cover having a protrusion that compresses organoid onto retention feature such that retention feature engages with organoid to retain it in place. The compression mechanism may ensure stable organoid positioning during measurement operations while preventing organoid displacement that could affect measurement quality, enabling consistent electrophysiological recordings across multiple measurement sessions.

Any of the systems or apparatuses disclosed herein may have any of the features (e.g., any of the probes, shanks, or computerized methods) of U.S. patent application Ser. No. 18/899,388, filed Sep. 27, 2024, entitled “DEVICES, SYSTEMS, AND METHODS FOR ORGANOID ANALYSIS” and incorporated by reference herein in its entirety.

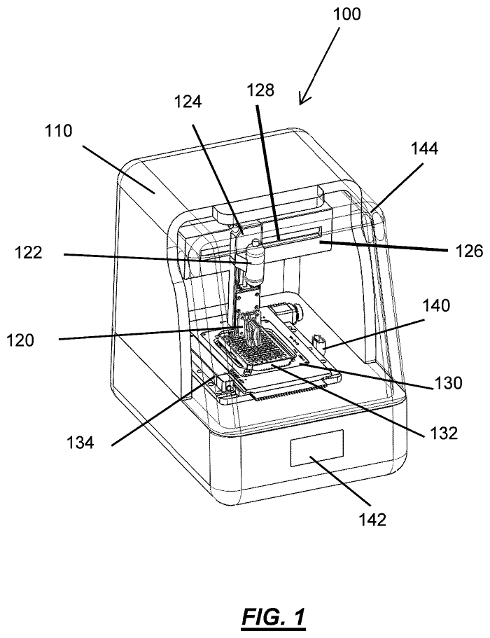

Referring to FIG. 1, a system 100 for probing organoids may include an enclosure 110, a probe head 120, a camera 122, a first stage 124, a track 126, a slot 128, a platen 130, a microwell plate 132, a second stage 134, a washing station 140, a display 142, and a door 144. System 100 may be configured as a high-throughput electrophysiology instrument with closed-loop feedback capabilities for automatically probing multiple organoids contained within microwell plate 132. System 100 may be a multi-stage instrument with closed-loop feedback to navigate and successfully record multiple electrophysiological activity from brain organoids contained in, for example, a 96-well titer, or microwell, plate, over a user defined period of time.

Enclosure 110 may provide environmental control for system 100, including control of temperature (e.g., minimum or maximum), CO2 levels, and humidity levels. In some cases, enclosure 110 acts as a faraday cage to shield probe head 120 and other internal components from ambient electromagnetic noise generated outside of system 100. Enclosure 110 may have one or more compartments defined within enclosure 110 to shield probe head 120 from noise generated by electronics and power supplies that operate system 100. In some cases, enclosure 110 may be designed to shield electromechanical components from damage due to user-defined environmental conditions. The enclosure may be designed in such a way to shield electromechanical components from damage due to user defined environmental conditions.

Door 144 may be coupled to enclosure 110 and may be constructed to further shield probe head 120 from outside noise. In some cases, door 144 may include or consist of a port configured for a user to insert microwell plate 132, similar to inserting a VHS tape into a VHS port. System 100 may be configured to sense insertion of microwell plate 132, grab microwell plate 132, and load microwell plate 132 into position for recording. In such an example, microwell plate 132 may include two or more titer plates in a single stack. This stack of titer plates may form a cartridge that is then placed on a loading port. System 100 may be configured to remove one titer plate at a time from the cartridge for recording. Display 142 may be integrated into enclosure 110 and may serve as a point of operation for a user. Display 142 may be a touch screen and enclosure 110 may include buttons or actuators to enable use of display 142 and enable user input.

Probe head 120 may have a probe and may be configured to insert the probe into a microwell of microwell plate 132 in order to measure a property of an organoid contained within the microwell. Microwell plate 132 may have a plurality of microwells, with each microwell containing an organoid. Probe head 120 may be automatically moved to probe organoids within microwell plate 132 through coordinated movement of first stage 124 and second stage 134.

First stage 124 may be configured such that probe head 120 is movable along first stage 124. Track 126 may be coupled to first stage 124 and may guide movement of probe head 120. Slot 128 may be defined within track 126 to accommodate movement of probe head 120 along first stage 124. Second stage 134 may be configured to move microwell plate 132, which may be positioned on platen 130. In some cases, first stage 124 and second stage 134 may be driven by a closed-loop feedback system based on electrophysiology data gathered from probe head 120. System 100 may comprise multiple linear stages to translate the instrument for multi-well recordings. The stages may be driven by a closed-loop feedback system based on electrophysiology data (e.g., spike data) gathered from the instrument tool. System 100 may also comprise rotational stages to provide additional freedom for probe positioning.

Camera 122 may be positioned within system 100 to aid in alignment and visualization during organoid probing operations. Camera 122 may use fiducials on microwell plate 132 and probe head 120 to precisely locate microwells for accurate probe insertion. Washing station 140 may contain a fluid for washing the probe of probe head 120 between measurements to clean organic material from the probe and inhibit cross contamination between different organoids.

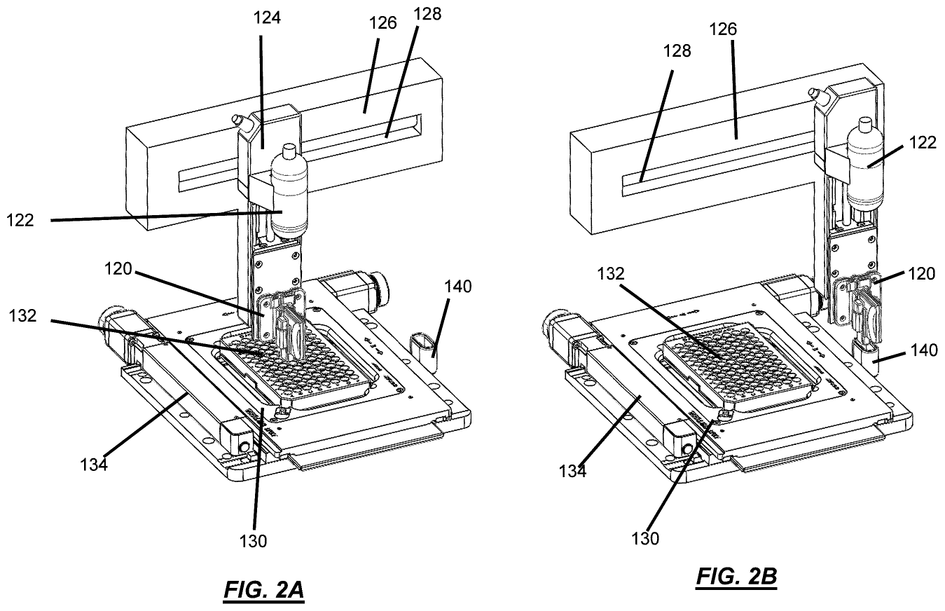

Referring to FIG. 2A and FIG. 2B, the stage system of system 100 may provide precise multi-axis motion control for automated organoid probing operations. Probe head 120 may be mounted on first stage 124, which may be configured as a high-resolution linear stage. A first stage 124 may enable movement of probe head 120 in an X direction along track 126. Track 126 may guide probe head 120 through slot 128 during X-axis translation, allowing probe head 120 to access different positions across microwell plate 132. As explained above and below, system 100 is comprised of a high-resolution XY stage fixed to the instrument floor. A titer plate is secured to this stage for navigating multiple wells. The instrument tool is secured to a high-resolution z-stage for controlled insertion. The z-stage is mounted to another X stage that serves to translate the instrument tool to a washing station.

Microwell plate 132 may be positioned on a platen 130, which may be mounted to second stage 134. A platen 130 may serve to precisely locate microwell plate 132 relative to a fixed position through tight machining tolerances, spring loaded mechanisms, or controller actuation. Second stage 134 may be configured as a high-resolution linear stage that enables movement of microwell plate 132 in a Y direction. The combination of X-direction movement of probe head 120 and Y-direction movement of microwell plate 132 may allow system 100 to position probe head 120 over any microwell within microwell plate 132.

With continued reference to FIG. 2A and FIG. 2B, probe head 120 may also be movable in a Z direction to raise or lower probe head 120. Z-direction movement may allow probe head 120 to insert a probe into an organoid within a microwell or retract probe head 120 away from the organoid. The Z-axis motion may be controlled with high precision to achieve controlled insertion depths and prevent damage to organoids during probing operations.

First stage 124 may be mounted to another X stage that serves to translate probe head 120 to washing station 140. This additional X-axis stage may allow probe head 120 to move between microwell plate 132 and washing station 140 for cleaning operations between measurements. Camera 122 may be positioned to provide visualization during stage movements and may assist with alignment of probe head 120 relative to microwells within microwell plate 132.

Alternative instrument configurations may comprise probe head 120 fixed to an XYZ stage suspended over an anchored microwell plate 132. In such configurations, an XYZ stage may be secured to microwell plate 132 positioned beneath a hanging probe head 120. Additional stages may be implemented to incorporate features such as automated probe loading, automated plate loading, and simultaneous well recordings using multiple probe heads 120 for parallel organoid analysis. Other instrument configurations may comprise of an instrument tool fixed to an XYZ stage suspended over an anchored titer plate such that the instrument tool is movable relative to the anchored titer plate. Inversely, an XYZ stage may be secured to a titer plate positioned beneath a hanging, or non-moving instrument tool such that the titer plate is movable relative to the instrument tool.

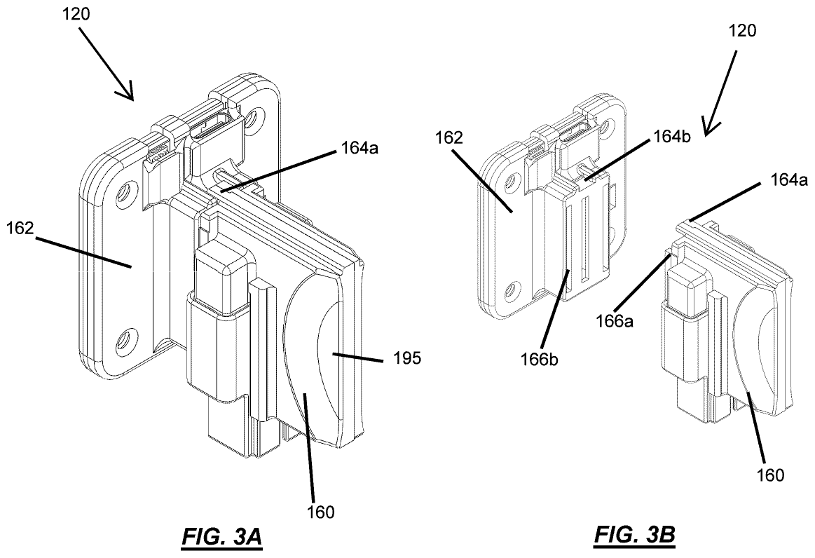

Referring to FIG. 3A and FIG. 3B, probe head 120 may include a tool 160, a motherboard housing 162, alignment features 164a and 164b, alignment features 166a and 166b, and a tool housing 195. Probe head 120 may be secured to a z-stage for insertion operations and may be comprised of multiple components that work together to provide precise organoid probing capabilities. Probe head 120 may be secured to a z-stage for insertion. Probe head 120 may be comprised of two components: the motherboard and the tool. Both components may be encapsulated inside of a cover or a case.

Tool 160 may be configured as a limited-use consumable intended to be easily removed and replaced. Tool 160 may be designed for single-use or limited-use applications to maintain measurement accuracy and prevent cross-contamination between different organoid samples. The consumable nature of tool 160 may allow for cost-effective operation while ensuring measurement quality across multiple probing sessions. The tool may be a limited-use consumable intended to be easily removed and replaced.

As shown in FIG. 3A and FIG. 3B, probe head 120 may further comprise motherboard housing 162 and tool housing 195 that are coupled to one another. Motherboard housing 162 may encapsulate the motherboard component, while tool housing 195 may encapsulate tool 160. The housings may prevent tampering while also protecting probe head 120 from environmental conditions and damage due to handling. The cover/case may prevent tampering while also protecting the probe head from environmental conditions and damage due to handling.

The motherboard and the probe board may be coupled to one another approximately orthogonally, providing a compact and stable configuration for probe head 120. The probe may extend from the probe board, allowing the probe to be positioned for insertion into organoids within microwell plate 132. The motherboard may be configured for single use or for long-term, repeated use.

The motherboard may include a connector for receiving the probe board. In some cases, the connector may be an edge connector that provides reliable electrical connection between the motherboard and probe board. The edge connector configuration may facilitate easy insertion and removal of the probe board, supporting the consumable design of tool 160.

Alignment features 164a and 164b may be positioned on motherboard housing 162 to ensure proper and precise coupling to the z-stage. Additional alignment features 166a and 166b may be positioned between motherboard housing 162 and tool housing 195 to ensure tool 160 is precisely located relative to the motherboard. These alignment features may provide mechanical registration that maintains accurate positioning during probe insertion operations. Alignment features on the motherboard may assist in ensuring proper and precise coupling to the z-stage. Additional alignment features on the motherboard and tool cases may assist in ensuring the tool is precisely located relative to the motherboard.

A cover may be coupled to tool housing 195, wherein the cover may be configured for covering the probe in at least some configurations. The cover may serve to protect the probe tip from damage due to handling and may be designed to accommodate different operational modes of probe head 120. The motherboard may be configured for single use or for long-term, repeated use, depending on the specific application requirements and operational protocols of system 100.

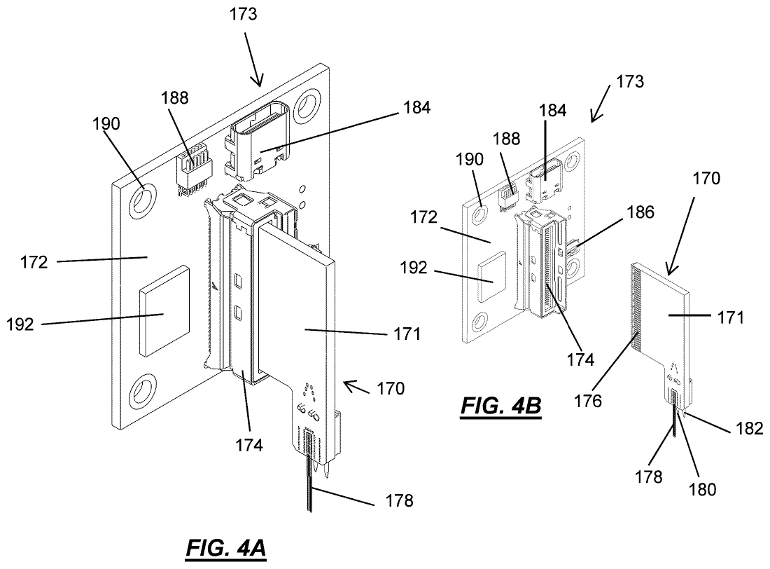

Referring to FIG. 4A and FIG. 4B, probe head 120 may include internal components that provide electrical connections, signal processing, and data transmission capabilities. A probe assembly 170 may include a probe board 171 and a probe 178. A motherboard assembly 173 may include a motherboard 172 with various electronic components for signal processing and control functions. Electrophysiology data may be recorded from a distal end of the tool, transmitted via an analog signal to the motherboard, where it is digitized and exported to the data acquisition or processing system housed in system 100 of FIG. 1 or to an external data acquisition or processing system electronically coupled to system 100.

Probe board 171 may be configured to support probe 178 and provide electrical connections between probe 178 and motherboard 172. A probe board connector 176 may be positioned on probe board 171 to establish electrical connection with motherboard 172. Probe 178 may extend from probe board 171 and may be configured to insert into organoids within microwell plate 132 for electrophysiological measurements.

With continued reference to FIG. 4A and FIG. 4B, motherboard 172 may include several components for signal processing and system control. A motherboard connector 174 may be positioned on motherboard 172 to receive probe board connector 176, establishing electrical communication between probe board 171 and motherboard 172. In some cases, motherboard connector 174 may be configured as an edge connector that provides reliable electrical connection. Alternative connectors may be implemented in place of the edge connector, such as Samtec™, Omnetics™, or other commonly used connectors.

Motherboard 172 may include an integrated circuit 192 that processes electrical signals received from probe 178. Integrated circuit 192 may be configured as an application-specific integrated circuit (ASIC) that digitizes analog signals from probe 178 and prepares the signals for transmission to a data acquisition system. An output 188 may be positioned on motherboard 172 to provide digital signal transmission from integrated circuit 192 to external data processing systems.

As shown in FIG. 4A and FIG. 4B, motherboard 172 may include a connector 184 configured for stimulation functions. Connector 184 may be a micro-USB connector that enables electrical stimulation of organoids during measurement operations. A switch 186 may be positioned on motherboard 172 to control activation of stimulation functions. Switch 186 may be configured as a single prong switch for stimulation on/off control, allowing users to selectively activate or deactivate stimulation capabilities during organoid probing operations.

Motherboard 172 may include mounting features 190 that provide mechanical attachment points for probe head 120. The mounting features 190 may be configured as grounded alignment features that ensure proper positioning of motherboard 172 within motherboard housing 162. In some cases, motherboard 172 may consist of four grounded alignment features 190, integrated circuit 192, output 188, connector 184, switch 186, and motherboard connector 174 configured to receive a 64-channel edge connector.

With continued reference to FIG. 4A and FIG. 4B, probe assembly 170 may include a ground wire 180 and a ref wire 182 that provide reference connections for electrical measurements. Ground wire 180 and ref wire 182 may be configured to enter a microwell above an organoid but within media to optimize quality of recording data. The positioning of ground wire 180 and ref wire 182 within the media may provide stable electrical reference conditions for accurate electrophysiological measurements.

Electrophysiology data may be recorded from a distal end of probe 178 and transmitted via analog signals to motherboard 172, where the signals are digitized by integrated circuit 192 and exported through output 188 to a data acquisition or processing system housed within system 100 or to an external data acquisition or processing system. Recording microelectrodes located on a shank of probe 178 may also be used to stimulate organoids, providing both measurement and stimulation capabilities through the same probe structure. The motherboard may include a controller for stimulating the organoid, with switch 186 providing user control for activating stimulation of the organoid during measurement operations.

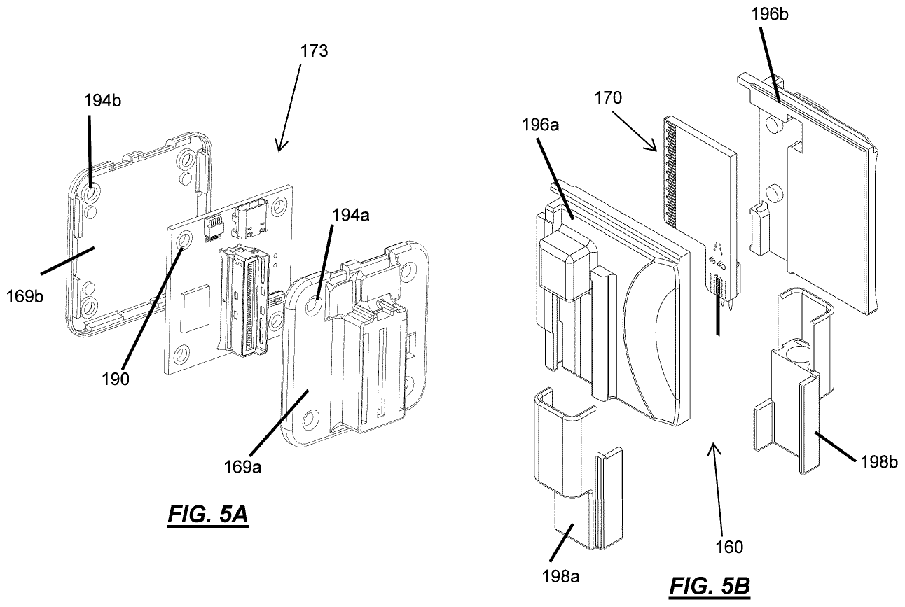

Probe head 120, discussed above with reference to FIGS. 3A and 3B, may be assembled from multiple housing components that provide protection and precise alignment for internal electronic components. For example, referring to FIG. 5A and FIG. 5B, a first piece of motherboard housing 169a and a second piece of motherboard housing 169b may be configured to enclose motherboard assembly 173 and provide mechanical protection during operation. The first piece of motherboard housing 169a and the second piece of motherboard housing 169b may be designed to mate together and form a complete enclosure around motherboard assembly 173. Motherboard assembly 173 may be positioned within the housing formed by first piece of motherboard housing 169a and second piece of motherboard housing 169b Integrated circuit 192 may be mounted on motherboard assembly 173 and may be protected by the housing structure during probe head 120 operations. The housing configuration may shield integrated circuit 192 from environmental conditions and mechanical damage while maintaining access to electrical connections.

With continued reference to FIG. 5A and FIG. 5B, a first housing mounting feature 194a and a second housing mounting feature 194b may be positioned on the housing components to provide secure attachment points. The first housing mounting feature 194a and the second housing mounting feature 194b may ensure proper alignment and mechanical coupling between first piece of motherboard housing 169a and second piece of motherboard housing 169b. These mounting features may provide precise registration that maintains accurate positioning of internal components during assembly and operation. These mounting features may align with mounting features 190 of motherboard 172, described above with reference to FIGS. 4A and 4B.

Tool 160 may be enclosed within a first tool housing 196a and a second tool housing 196b that provide protection for probe assembly 170. The first tool housing 196a and the second tool housing 196b may be configured to mate together and form a complete enclosure around tool 160. Probe assembly 170 may be positioned within the housing formed by first tool housing 196a and second tool housing 196b, providing mechanical protection for probe components during handling and operation. The tool may be comprised of a probe assembly, probe case, and a spring-loaded, telescopic cover which serves to protect the probe tip from damage due to handling.

As shown in FIG. 5A and FIG. 5B, a first cover 198a and a second cover 198b may be coupled to first tool housing 196a and second tool housing 196b respectively. The first cover 198a and the second cover 198b may be configured as telescopic covers that serve to protect probe tips from damage due to handling. The telescopic design may allow first cover 198a and second cover 198b to extend and retract, providing probe protection during storage and transport while allowing probe access during measurement operations. The probe case may have a ‘grip’ feature to make insertion and removal from instrument easier.

The first cover 198a and the second cover 198b may be spring-loaded to provide automatic extension and retraction capabilities. The spring-loaded mechanism may ensure that probe tips remain protected when not in use while allowing easy access during organoid probing operations. The telescopic cover mechanism may be designed to accommodate different probe lengths and configurations while maintaining consistent protection levels.

With continued reference to FIG. 5A and FIG. 5B, first tool housing 196a and second tool housing 196b may include grip features that facilitate insertion and removal of tool 160 from probe head 120. The grip features may be configured as textured surfaces, raised areas, or contoured shapes that provide enhanced manual handling capabilities. These grip features may make insertion and removal of tool 160 from the instrument easier for users during tool replacement operations.

In some cases, first cover 198a and second cover 198b may be comprised of a conductive material that doubles as a faraday cage to shield probe 178 during recordings. The conductive material may provide electromagnetic shielding that reduces electrical noise and interference during electrophysiological measurements. The grounded cover configuration may enhance signal quality by creating an electromagnetic barrier around probe 178, improving measurement accuracy and reducing artifacts in recorded data. Alternative concepts may include a removable cover, configured to be removed prior to tool assembly to probe head; a fixed cover that fits inside the ‘negative’ space between wells; and a grounded cover, comprised of a conductive material, that doubles as a faraday cage to shield the probe during recordings.

In some aspects, probe head 120 may include an integrated camera. The endoscopic camera integration may provide visual guidance for probe alignment relative to organoids within microwell plate 132 (of FIGS. 1, 2A, and 2B).

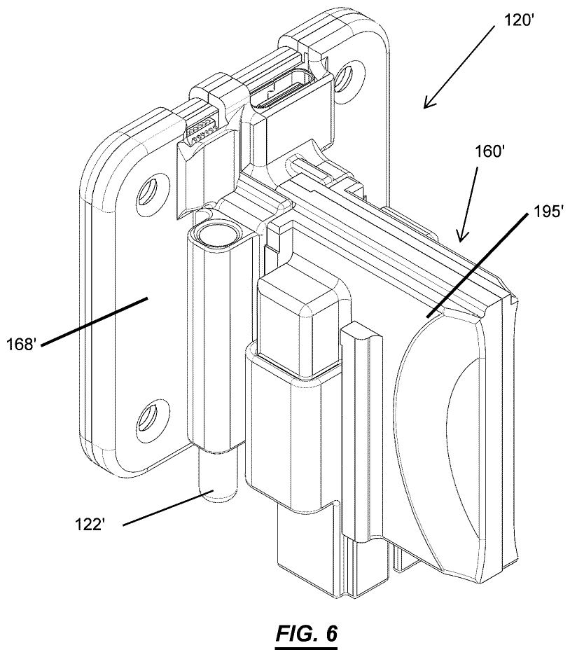

Referring now to FIG. 6, variations of the integrated camera configuration may include a camera 122′, a probe head 120′, a tool 160′, a motherboard housing 168′, and a tool housing 195′. These alternative configurations may provide different camera positioning options, housing geometries, or tool configurations to accommodate specific measurement requirements or operational constraints. The variations may allow for customization of probe head 120 based on particular organoid types, microwell plate configurations, or experimental protocols. Camera 122′ may capture images that show the relationship between probe 178 (FIGS. 4A, 4B, and 5B) and organoid structures, allowing for precise positioning before and during probe insertion. The visual feedback from camera 122′ may be used in conjunction with closed-loop feedback systems to optimize probe placement and measurement quality. For example, the integrated camera configuration may aid with visualization and alignment during organoid insertion and may provide real-time feedback for precise probe positioning.

Camera 122′ may be positioned at a junction between tool housing 195′ and motherboard housing 168′ to provide enhanced visualization capabilities during organoid probing operations. Camera 122′ may be configured as an endoscopic camera that runs along an outside of probe head 120′. Camera 122′ may be positioned to capture images of the probe insertion area, allowing operators to monitor probe alignment and organoid positioning in real-time.

As shown in FIG. 6, motherboard housing 168′ may be configured to accommodate camera 122′ while maintaining the structural integrity and electromagnetic shielding properties of probe head 120′. The integration of camera 122′ into motherboard housing 168′ may provide a compact design that minimizes the overall footprint of probe head 120′ while adding visualization capabilities. Tool housing 195′ may be designed to work in conjunction with motherboard housing 168′ to provide optimal camera positioning and field of view.

Camera 122′ may include quick connect features that allow for data transmission from camera 122′ to an instrument computer within system 100. The quick connect configuration may facilitate easy connection and disconnection of camera 122′ during maintenance operations or when replacing tool 160′. Data transmission from camera 122′ may be synchronized with electrophysiological data collection to provide coordinated visual and electrical recordings of organoid responses. An endoscopic camera running along the PCB aiming towards the probe tips to serve as a visual aid for alignment and organoid insertion. Similarly, this configuration may include a quick connect to allow for data transmission of the camera to the instrument computer.

The junction positioning of camera 122′ between tool housing 195′ and motherboard housing 168′ may provide an optimal viewing angle for organoid visualization while maintaining the mechanical stability of probe head 120′. The camera positioning may allow for clear visualization of probe tips and organoid surfaces without interfering with probe insertion operations or electrical measurements. Camera 122′ may be oriented to provide a field of view that encompasses both the probe insertion area and surrounding microwell structures for comprehensive visual monitoring.

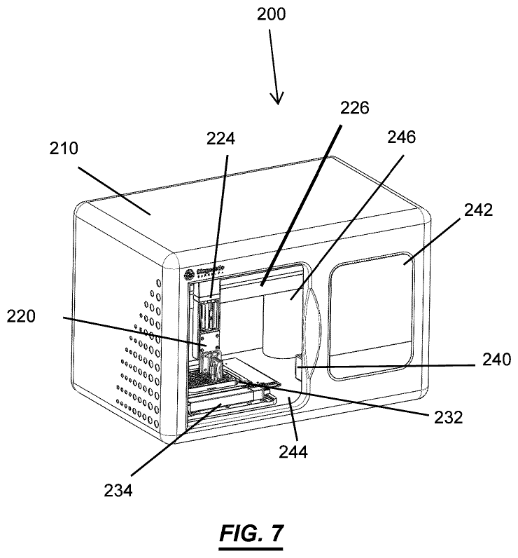

Referring to FIG. 7, an alternative high-throughput system 200 may include an enclosure 210, a probe head 220, a first stage 224, a track 226, a microwell plate 232, a second stage 234, a washing station 240, a display 242, a door 244, and a fluid reservoir 246. System 200 may be configured as an alternative high-throughput electrophysiology instrument that incorporates enhanced fluid handling capabilities for media exchange, drug delivery, and probe washing operations during automated organoid probing. System 100 may have any or all of the characteristics of system 100, except as described below.

Enclosure 210 may provide environmental control functions similar to enclosure 110, including temperature regulation, CO2 control, and humidity management. Enclosure 210 may also act as a faraday cage to shield probe head 220 and internal components from electromagnetic interference. Door 244 may be coupled to enclosure 210 and may provide access to internal components while maintaining environmental isolation during operation. Display 242 may be integrated into enclosure 210 to provide user interface capabilities for system control and monitoring.

With continued reference to FIG. 7, probe head 220 may be configured with integrated fluid handling capabilities. Probe head 220 may have any feature of probe head 120 unless otherwise specified. Probe head 220 may include one or more fluid port tips running along a printed circuit board towards a probe for media exchange, drug delivery, and probe washing operations. The fluid port tips may be positioned to deliver fluids directly to microwell locations during organoid probing operations, enabling real-time media replenishment and targeted drug delivery.

Probe head 220 may have a quick connect feature to allow for fluid transport from fluid reservoir 246 housed within enclosure 210. The quick connect configuration may facilitate rapid connection and disconnection of fluid lines during maintenance operations or when replacing consumable components of probe head 220. In other words, the quick connect system may provide reliable fluid connections while maintaining the ability to easily service and replace probe components.

As shown in FIG. 7, fluid reservoir 246 may be positioned within enclosure 210 and may contain a nourishing media for organoids within microwell plate 232. Fluid reservoir 246 may be configured to supply media through fluid lines connected to probe head 220 via the quick connect system. The nourishing media may be formulated to maintain organoid health and viability during extended recording periods, providing nutrients and maintaining appropriate chemical conditions for organoid survival. Depending on the environmental settings configured into the enclosure, and the length of the recording, additional media may be deposited into the wells to maintain the health of the organoids.

First stage 224 may be configured to provide X-direction movement of probe head 220 along track 226, similar to the configuration described for system 100. Second stage 234 may be configured to provide Y-direction movement of microwell plate 232, enabling precise positioning of probe head 220 relative to individual microwells within microwell plate 232. The combination of first stage 224 and second stage 234 may provide coordinated multi-axis motion control for automated organoid probing operations.

With continued reference to FIG. 7, washing station 240 may be configured to work in conjunction with the fluid handling capabilities of probe head 220. Washing station 240 may receive cleaning fluids from fluid reservoir 246 or from separate cleaning fluid reservoirs within enclosure 210. The integrated fluid system may enable automated probe cleaning operations between measurements, reducing cross-contamination and maintaining measurement accuracy across multiple organoid samples.

System 200 may provide adaptive media replenishment capabilities based on environmental settings configured within enclosure 210 and the length of recording operations. Additional media may be deposited into microwells of microwell plate 232 to maintain organoid health during extended measurement sessions. The media exchange may occur via fluid port tips built into probe head 220 or through independently actuated mechanisms controlled by system 200. This media exchange may occur via a fluid port built into the probe head or through independently actuated/controlled mechanism.

The fluid handling capabilities of system 200 may enable drug delivery operations during organoid probing. Drug delivery mechanisms may operate similarly to media exchange operations, utilizing the same fluid port tips and quick connect systems integrated into probe head 220. The drug delivery system may provide precise control over drug concentration and timing, enabling pharmacological studies and therapeutic screening applications using organoid models. The drug delivery mechanism may be the same or similar to the operations previously described with regards to a media exchange.

As shown in FIG. 7, the integration of fluid reservoir 246 within enclosure 210 may provide a compact and controlled environment for fluid storage and delivery. Fluid reservoir 246 may be configured with temperature control, mixing capabilities, and contamination prevention features to maintain fluid quality during operation. The reservoir system may support multiple fluid types simultaneously, enabling complex experimental protocols that require sequential or simultaneous delivery of different media compositions or drug formulations.

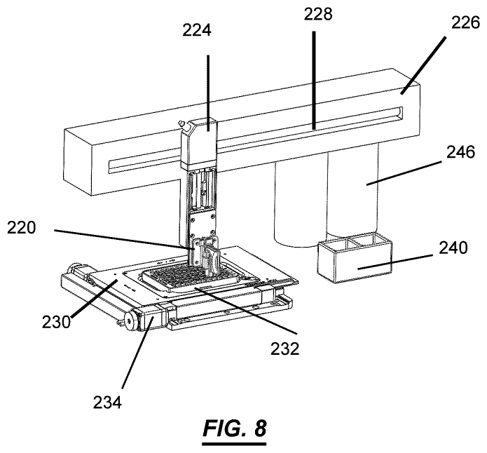

Referring to FIG. 8, the stage system of system 200 may provide enhanced motion control and fluid delivery capabilities through an integrated configuration of mechanical and fluidic components. A probe head 220 may be mounted on a first stage 224, which may be configured to provide precise linear motion along a track 226. Track 226 may include a slot 228 that accommodates movement of probe head 220 during X-axis translation operations, allowing probe head 220 to access different positions across microwell plate 232.

A camera may be positioned within system 200 to provide visual monitoring and alignment capabilities during automated organoid probing operations. The camera may be viewing the microwell plate from above, or may be mounted to view from below. The camera may be configured to capture images of microwell plate 232 and probe head 220 positioning, enabling real-time feedback for motion control systems. The camera may work in conjunction with first stage 224 to provide precise positioning feedback during probe alignment and insertion operations.

A microwell plate 232 may be positioned on a platen 230, which may be mounted to second stage 234. Platen 230 may serve to precisely locate microwell plate 232 relative to a fixed reference position through mechanical registration features, spring-loaded mechanisms, or active positioning control. Second stage 234 may be configured to provide Y-direction movement of microwell plate 232, enabling coordinated multi-axis positioning when combined with X-direction movement of probe head 220 along first stage 224.

The motion control system may integrate feedback from camera with position data from first stage 224 and second stage 234 to achieve precise probe positioning relative to individual microwells within microwell plate 232. The coordinated motion control may enable automated scanning of multiple organoids within microwell plate 232 while maintaining consistent probe insertion parameters and measurement quality across different microwell locations.

As shown in FIG. 8, washing station 240 may be positioned within the motion envelope of first stage 224, allowing probe head 220 to access washing station 240 during automated cleaning operations. Washing station 240 may be integrated with fluid reservoir 246 to provide automated probe cleaning capabilities between measurements. The washing station may be built into microwell plate 232 by leveraging unused space around a perimeter of microwells, providing a compact cleaning solution that does not require additional motion axes or workspace. Alternatively, the cleaning station may be built into a custom titer plate by leveraging some of the unused space around the perimeter of the microwells.

Fluid reservoir 246 may be connected to probe head 220 through fluid lines that accommodate movement of probe head 220 along first stage 224 and track 226. The fluid delivery system may provide media exchange, drug delivery, and probe washing capabilities through integrated fluid port tips positioned on probe head 220. The fluid lines may be configured with sufficient flexibility and length to accommodate the full range of motion of probe head 220 without restricting movement or creating mechanical interference. Additionally or alternatively, a washing mechanism may be integrated into the tool. In this instance, the probe head may travel over a waste container. Cleaning solution would flow over the probe tips and into the waste dispenser.

With continued reference to FIG. 8, a fluid transport mechanism may be designed into microwell plate 232 and platen 230 to allow for drug delivery without actuation. The passive drug delivery system may utilize microfluidic channels, capillary action, or diffusion-based transport to deliver drugs or other compounds to organoids within microwells. The passive delivery mechanism may eliminate the need for active pumping or actuation systems while providing controlled delivery of compounds to organoid samples. Alternatively, a fluid transport mechanism may be designed into the microwell plate and platen to allow for drug delivery without actuation.

The integrated fluid transport mechanism within microwell plate 232 may include channels that connect to fluid reservoir 246 through platen 230, enabling continuous or controlled delivery of media or drugs to individual microwells. The passive delivery system may provide consistent compound concentrations across multiple microwells while reducing system complexity and potential sources of mechanical failure. The fluid transport mechanism may be configured to work in conjunction with the active fluid delivery capabilities of probe head 220 to provide comprehensive fluid handling options for different experimental protocols.

System 200 may integrate third party stimulation devices. Such devices may include optical and drug delivery stimulation. Such integration may be accomplished via provided ports in the enclosure and/or a breadboard inside the enclosure that users can mount stimulation devices (e.g., LED light path or microfluidic dispenser for drug delivery, etc.) thereon. Electrical stimulation using a probe may also be utilized. Additionally, system 200 may be compatible with 3rd party automation platforms. Such platforms may include warehouse robots with pre-programmed trajectory mapping or similar functionality.

System 200 may comprise one or more cameras to aid in titer plate alignment and/or organoid visualization. A grin lens may be utilized as a toolhead. An electrode array may be attached to the lens to allow simultaneous optical (Ca+) imaging and electrophysiology in 3D cultures. Camera(s) may be installed at the platen to view the organoids from the bottom.

System 200 may include a UV light integrated into the environmental enclosure to provide sterilization. A localized or mobile UV light may be integrated within the instrument to provide precisely aimed sterilization. System 200 may include a user display for monitoring recording progress and configuring custom user settings. This display may be integrated into the enclosure (i.e. a tablet) or a standalone monitor/keyboard combo fixed to the enclosure by some means (e.g., permanently fixed or on an adjustable arm). Alternatively, the user display may be minimized with primary instrument controls/GUI. Additionally or alternatively, the system described herein may be plugged into a PC via a USB or other electrical connection.

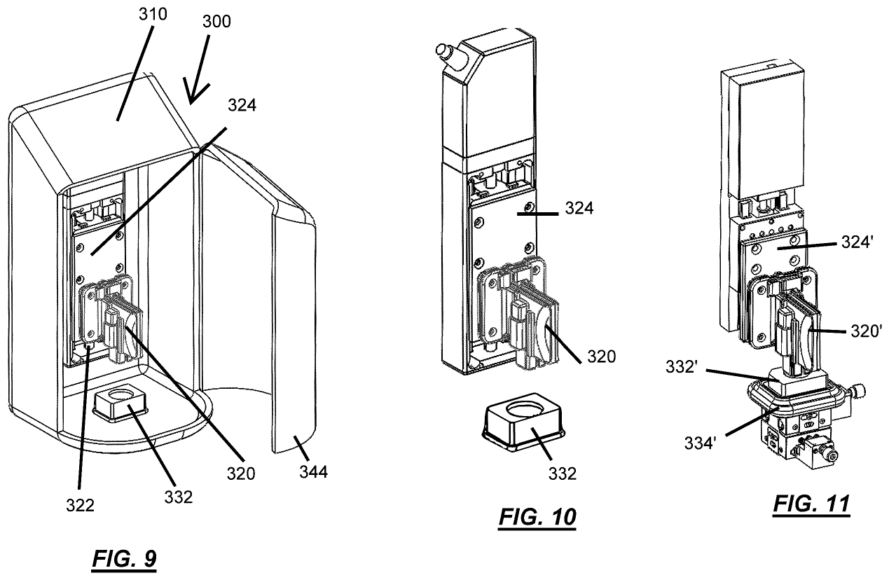

Referring to FIG. 9, FIG. 10, and FIG. 11, a system 300 may be configured as a low-throughput electrophysiology instrument designed for single organoid probing operations. System 300 may have any or all of the characteristics of systems 100 and 200, except as described below. System 300 may include an enclosure 310, a probe head 320 (having any property of probe head 120), a camera 322, a stage 324, a microwell plate 332, and a door 344. System 300 may be a single stage instrument with closed-loop z-feedback to successfully record electrophysiological activity from a single brain organoid contained in a titer plate over a user defined period.

Enclosure 310 may provide environmental control functions similar to those described for high-throughput systems, including temperature regulation, CO2 control, and humidity management. Enclosure 310 may act as a faraday cage to shield probe head 320 from electromagnetic interference during measurement operations. Door 344 may be coupled to enclosure 310 and may provide access to internal components while maintaining environmental isolation during organoid probing operations.

Probe head 320 may be configured with closed-loop z-feedback capabilities to record electrophysiological activity from a single organoid contained within microwell plate 332. Probe head 320 may be designed to operate over user-defined periods while maintaining measurement stability and accuracy. The closed-loop feedback system may enable automatic positioning and depth control during organoid insertion without requiring complex multi-axis automation systems.

Stage 324 may be configured to provide Z-direction movement of probe head 320 for controlled probe insertion and retraction operations. Stage 324 may include additional mechanical stages for fine-tuned XY positioning relative to the Z-stage movement. The manual XY alignment capability may allow users to precisely position probe head 320 relative to an organoid within microwell plate 332 before initiating automated Z-axis insertion operations. System 300 may include additional mechanical stages for finely located XY position relative to z-stage.

As shown in FIG. 9, FIG. 10, and FIG. 11, system 300 may lack automated XY or rotational alignment systems that are present in high-throughput configurations. Manual alignment may be utilized instead of automated positioning systems, providing users with direct control over probe positioning while reducing system complexity and cost. The manual alignment approach may be suitable for applications where precise user control is preferred over automated scanning capabilities.

Camera 322 may be positioned within system 300 to provide visualization capabilities during manual alignment and organoid probing operations. Camera 322 may assist users in positioning probe head 320 relative to organoids within microwell plate 332 during manual XY alignment procedures. The visual feedback from camera 322 may enable precise probe positioning before initiating automated Z-axis insertion operations.

With continued reference to FIG. 9, FIG. 10, and FIG. 11, microwell plate 332 may be configured to contain a single organoid for individual analysis operations. In some aspects, microwell plate 332 may include only one microwell, providing a simplified platform for focused organoid studies.

System 300 may include alternative configurations shown in FIG. 11 as probe head 320′, first stage 324′, second stage 334′, and microwell plate 332′. These alternative configurations may provide different positioning options, stage geometries, or microwell arrangements to accommodate specific measurement requirements or experimental protocols. The variations may allow for customization of system 300 based on particular organoid types or research applications. The configurations shown in FIG. 10 and in FIG. 11 may be used interchangeably with system 300, shown in FIG. 9.

As shown in FIG. 9, FIG. 10, and FIG. 11, system 300 may have minimal integrated display requirements compared to high-throughput systems. For example, users may connect to system 300 with a computer or other device to run operating software, eliminating the need for complex integrated display.

With continued reference to FIG. 9, FIG. 10, and FIG. 11, system 300 may be compatible with third-party stimulation devices and automation platforms. Compatibility may be achieved through provided ports in enclosure 310 or breadboard mounting systems that allow users to integrate additional equipment as needed. Such compatibility or combinations may be achieved via provided ports in the enclosure and/or a breadboard inside the enclosure that users can mount stimulation devices on (such as an LED light path or microfluidic dispenser for drug delivery). The modular design approach may provide flexibility for customizing system 300 capabilities based on specific research requirements while maintaining the core low-throughput functionality.

For example, system 300 may be configured to be compatible or combined with 3rd party stimulated devices (e.g., optical and drug delivery). Additionally or alternatively, the system may be compatible with 3rd party automation platforms (such as warehouse robots with pre-programmed trajectory mapping or similar functionality).

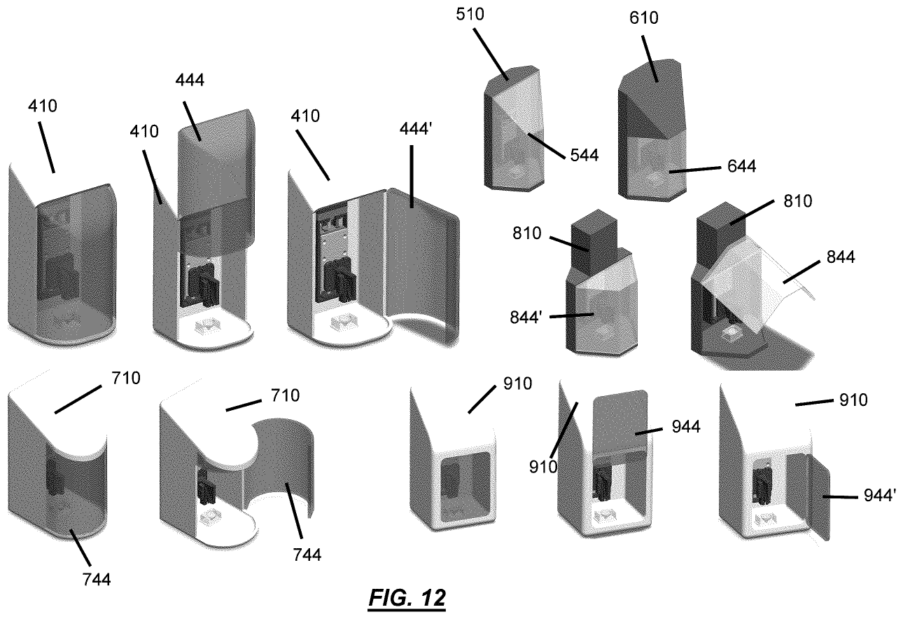

Referring to FIG. 12, various housing and door configurations may be implemented to provide different access mechanisms and operational capabilities for organoid probing systems. An enclosure 410 may include a door 444 and a door 444′ that provide alternative access configurations for system operation. The door 444′ may be configured as a hinged door that pivots relative to enclosure 410, while door 444 may be configured as a slidable door that translates relative to enclosure 410. The hinged and slidable door configurations may provide different operational advantages based on laboratory space constraints and user preferences.

With continued reference to FIG. 12, an enclosure 510 may include a door 544 that provides access to internal components while maintaining environmental isolation during operation. Door 544 may be configured with sealing mechanisms that preserve temperature, humidity, and CO2 conditions within enclosure 510 during organoid probing operations. The door configuration may be selected based on specific environmental control requirements and operational protocols for different organoid analysis applications.

An enclosure 610 may be configured with a relatively smaller door 644 that does not cover an entire front of enclosure 610 but is otherwise similar to door 544.

As shown in FIG. 12, an enclosure 710 may include a door 744 that provides alternative access configurations for different operational modes. Door 744 may be configured to accommodate different loading procedures, maintenance operations, or user interaction requirements based on specific system configurations. The door design may be optimized for particular applications while maintaining compatibility with environmental control and electromagnetic shielding requirements.

An enclosure 810 may include door 844 and door 844′that provide multiple access options for enhanced operational flexibility. The multiple door configuration may enable simultaneous access to different system components or provide redundant access capabilities for maintenance and operation procedures. Door 844 and door 844′ may be configured with different access mechanisms that optimize user interaction for specific operational requirements. Door 844 and door 844′ may be hinged or may include a sliding mechanism to enable access to internal components of enclosure 910.

With continued reference to FIG. 12, an enclosure 910 may include door 944 and door 944′ that provide alternative access configurations for different system components or operational modes. The dual door configuration may enable independent access to different system areas while maintaining environmental isolation for sensitive components. Door 944 and door 944′ may be configured with different sealing mechanisms, access procedures, or operational characteristics based on specific system requirements.

The various door and enclosure configurations shown in FIG. 12 may be selected based on specific operational requirements, laboratory constraints, or user preferences for different organoid probing applications. The door configurations may provide different levels of environmental control, electromagnetic shielding, or user access capabilities based on particular system requirements. The enclosure designs may accommodate different system sizes, component arrangements, or operational workflows while maintaining core functionality for organoid probing operations.

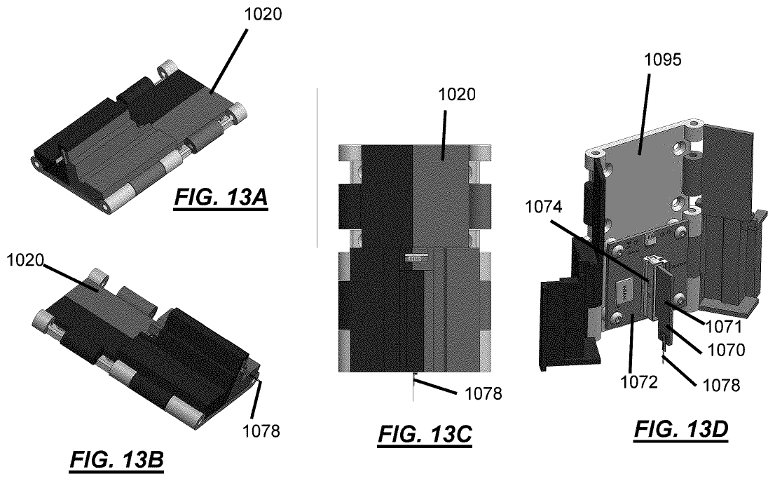

Referring to FIGS. 13A-13D, a probe head 1020 may be configured with an alternative housing design that provides enhanced protection and accessibility for probe components. Probe head 1020 may include a probe assembly 1070, a probe board 1071, a motherboard 1072, a motherboard connector 1074, a probe 1078, and a housing 1095. The alternative housing configuration may provide improved mechanical protection while maintaining accessibility for probe replacement and maintenance operations.

With continued reference to FIGS. 13A-13D, housing 1095 may be formed of one component having a plurality of panels hingedly coupled to one another. The hinged panel configuration may enable housing 1095 to be assembled around probe assembly 1070 while providing access for maintenance and component replacement operations. The single-component design with hinged panels may reduce manufacturing complexity while maintaining structural integrity and protection capabilities for internal components.

Probe assembly 1070 may be positioned within housing 1095 and may include probe board 1071 and probe 1078 configured for organoid measurement operations. Motherboard 1072 may be positioned within housing 1095 and may include motherboard connector 1074 for establishing electrical connection with probe board 1071. The integrated housing design may provide comprehensive protection for both probe assembly 1070 and motherboard 1072 while maintaining electrical connectivity and operational functionality.

As shown in FIGS. 13A-13D, the hinged panel configuration of housing 1095 may enable the housing to be opened for component access while maintaining structural integrity during normal operation. The hinged panels may be configured with locking mechanisms that secure housing 1095 in the closed position during measurement operations while enabling easy opening for maintenance procedures. The panel design may accommodate different probe configurations and motherboard arrangements while providing consistent protection and accessibility.

The single-component housing design may reduce assembly complexity and potential failure points compared to multi-component housing configurations. The hinged panel approach may enable housing 1095 to be manufactured as a single piece that folds around internal components, eliminating the need for separate housing pieces and associated alignment features. The integrated design may provide consistent dimensional accuracy and mechanical stability while simplifying manufacturing and assembly procedures.

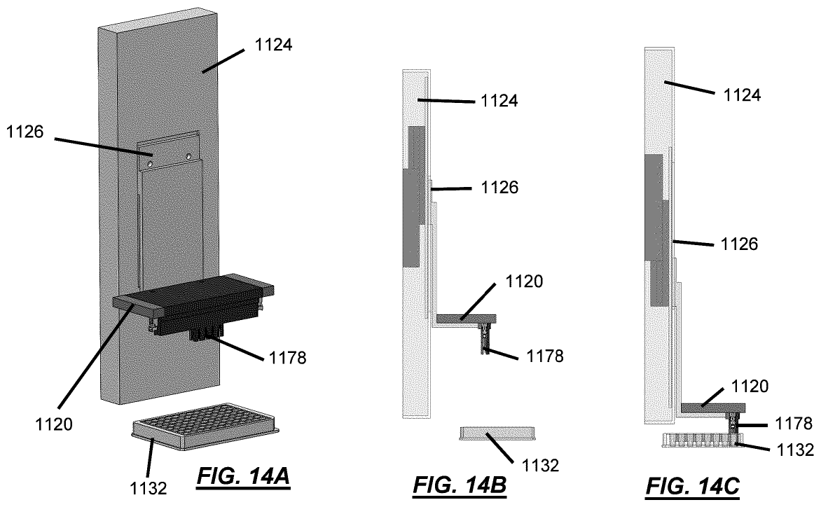

Referring to FIG. 14A, FIG. 14B, and FIG. 14C, a probe head 1120 may be configured with multiple probes 1178 for enhanced measurement capabilities during organoid analysis operations. Probe head 1120 may be mounted on a stage 1124 that provides vertical movement along a track 1126. A microwell plate 1132 may be positioned below probe head 1120 to contain organoids for measurement operations. The multi-probe configuration may enable simultaneous measurement from multiple organoids or enhanced spatial coverage within individual organoids.

With continued reference to FIG. 14A, FIG. 14B, and FIG. 14C, stage 1124 may be configured to provide a large vertical range of motion that enables probe head 1120 to move between different operational positions. The vertical gap between stage 1124 and a top of microwell plate 1132 may provide clearance for probe head 1120 movement during positioning and insertion operations. The large vertical range may accommodate different microwell plate configurations and enable probe head 1120 to access washing stations or other operational positions.

Track 1126 may provide mechanical guidance for probe head 1120 movement along stage 1124 while accommodating the vertical range of motion required for different operational modes. The track configuration may ensure precise positioning of probe head 1120 relative to microwell plate 1132 while providing sufficient clearance for probe insertion and retraction operations. The mechanical guidance system may maintain positioning accuracy throughout the full range of vertical motion.

As shown in FIG. 14A, FIG. 14B, and FIG. 14C, probes 1178 may be arranged in a linear configuration that enables systematic coverage of multiple microwells within microwell plate 1132. The linear arrangement may enable probe head 1120 to simultaneously access multiple organoids during measurement operations while maintaining independent positioning control for individual probes. The multi-probe configuration may increase measurement throughput while maintaining measurement quality for individual organoids.

The vertical stage configuration may enable probe head 1120 to move up and down along stage 1124 while maintaining precise positioning relative to microwell plate 1132. The up and down movement capability may enable probe head 1120 to insert probes 1178 into organoids within microwell plate 1132 or retract probes 1178 away from organoids based on operational requirements. The vertical motion control may provide precise depth control for probe insertion operations while accommodating different organoid sizes and microwell configurations.

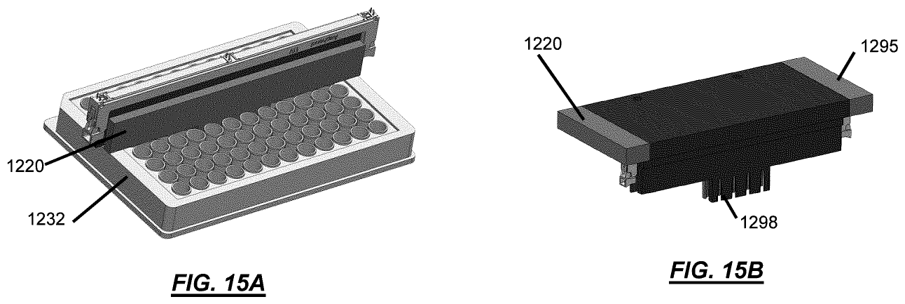

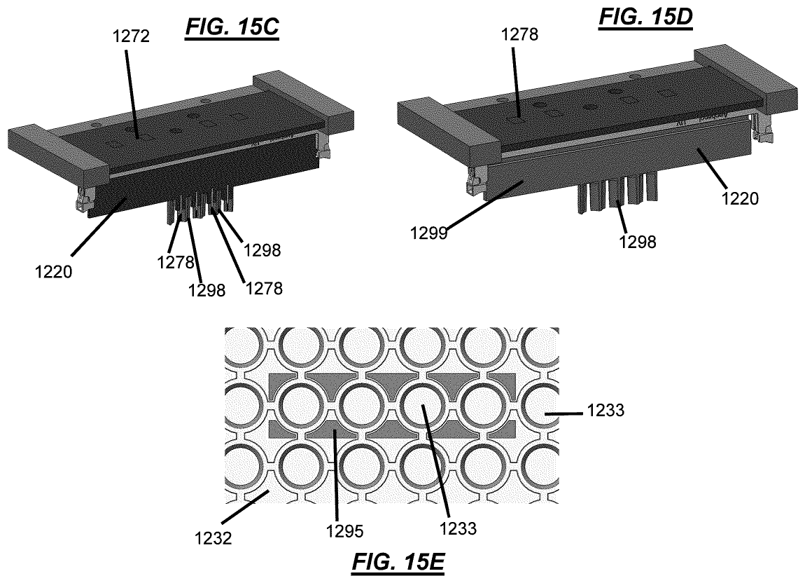

Referring to FIG. 15A and FIG. 15B, a probe head 1220 may be configured with a specialized cover 1298 that fits into negative spaces between wells of a microwell plate 1232. Probe head 1220 may include a housing 1295 that supports multiple probes for simultaneous organoid measurement operations. The cover 1298 configuration may provide enhanced positioning stability and contamination prevention during multi-well measurement procedures. For example, the shaped cover configuration may enable cover 1298 to fit precisely into the spaces between microwells while providing mechanical stability for probe head 1220 during measurement operations. The negative space fitting may prevent cross-contamination between adjacent microwells while maintaining precise probe positioning.

Housing 1295 may support multiple probes arranged in a linear configuration that corresponds to the microwell arrangement within microwell plate 1232. The linear probe arrangement may enable systematic coverage of multiple microwells while maintaining the negative space fitting capability of cover 1298. The housing configuration may provide structural support for the probe array while accommodating the specialized cover design.

As shown in FIG. 15A and FIG. 15B, the negative space fitting capability of cover 1298 may enable probe head 1220 to achieve precise positioning relative to microwell plate 1232 while providing mechanical stability during measurement operations. The cover configuration may prevent lateral movement of probe head 1220 during probe insertion procedures while maintaining access to individual microwells for measurement operations. The negative space fitting may provide passive alignment that ensures consistent probe positioning across multiple measurement sessions.

Referring to FIGS. 15C-15E, probe head 1220 may include additional features that enhance the negative space fitting capability and measurement performance. A well 1233 may be shown in relation to probe head 1220 components including a motherboard 1272, probes 1278, a housing 1295, a cover 1298, and a back 1299. The detailed configuration may illustrate how the negative space fitting system works in conjunction with probe positioning and measurement capabilities.

Probe head 1220 may include a plurality of probes 1278 arranged linearly to correspond with the microwell arrangement of microwell plate 1232. Each probe of probes 1278 may be associated with a respective cover that has the shape corresponding to the shape of the negative space between adjacent microwells. The multiple cover configuration may provide individual negative space fitting for each probe while maintaining the overall linear arrangement of the probe array.

Motherboard 1272 may be positioned within housing 1295 to provide electrical connections and signal processing capabilities for probes 1278. The motherboard configuration may support the multi-probe array while maintaining compatibility with the negative space fitting cover system. Back 1299 may provide structural support for the probe head assembly while accommodating the specialized cover configuration and probe arrangement.

As shown in FIGS. 15C-15E, the negative space fitting system may provide enhanced contamination prevention by creating physical barriers between adjacent microwells during measurement operations. The cover configuration may prevent fluid transfer between microwells while maintaining individual probe access to organoids within each microwell. The contamination prevention capability may be particularly important for multi-well measurement operations where cross-contamination could affect measurement accuracy or organoid viability.

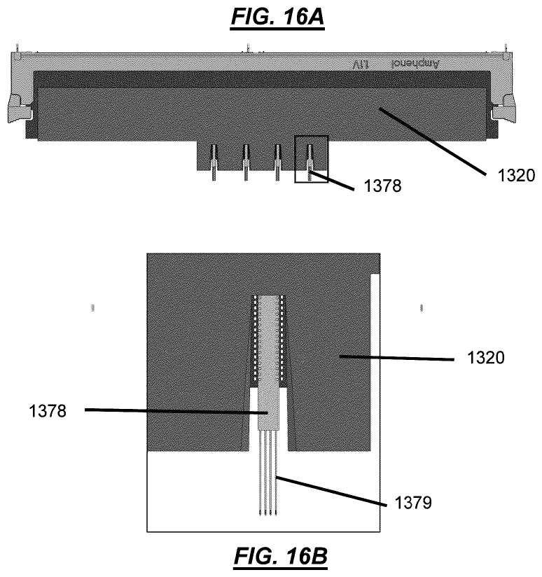

Referring to FIG. 16A and FIG. 16B, a probe head 1320 may be configured with probes 1378 that include multiple shanks 1379 for enhanced measurement capabilities. FIG. 16B is a detailed view of probe 1378, shown in FIG. 16A. Each probe of probes 1378 may include a plurality of shanks 1379 that provide multiple recording sites within individual organoids. The multi-shank configuration may enable comprehensive spatial coverage of organoid electrical activity while maintaining compatibility with the probe head positioning and control systems.

Each probe of probes 1378 may include at least four shanks 1379, providing multiple recording sites that enable detailed analysis of organoid electrical activity patterns. The four-shank configuration may provide balanced spatial coverage while maintaining probe structural integrity and insertion capabilities. Each shank of shanks 1379 may include a plurality of channels that provide multiple recording sites along the length of each shank.

The multi-shank probe configuration may enable simultaneous recording from multiple locations within individual organoids while maintaining the ability to probe multiple organoids through the multi-probe array of probe head 1320. The combination of multiple probes with multiple shanks per probe may provide comprehensive spatial and temporal coverage of organoid electrical activity across multiple organoid samples during simultaneous measurement operations.

As shown in FIG. 16A and FIG. 16B, the shank configuration may be optimized to provide maximum recording coverage while maintaining probe insertion capabilities and structural integrity. The shanks may be arranged to provide systematic coverage of organoid tissue while minimizing mechanical damage during insertion procedures. The multi-channel capability of each shank may enable detailed analysis of electrical activity patterns within organoid tissue while maintaining compatibility with automated positioning and insertion systems.

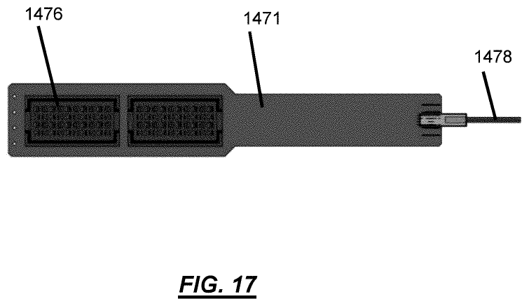

Referring to FIG. 17, a probe board 1471 may be configured with alternative connector systems that provide enhanced electrical connectivity and mechanical stability. Probe board 1471 may include a probe board connector 1476 and a probe 1478 configured for organoid measurement operations. The probe board connector 1476 may be configured as a Samtec™ connector, or an alternative connector, for connecting to a motherboard connector, providing reliable electrical connection with enhanced mechanical stability compared to standard edge connectors.

With continued reference to FIG. 17, the connector configuration may provide enhanced electrical performance and mechanical reliability compared to edge connector systems. The connector may provide multiple contact points that ensure reliable electrical connection while accommodating mechanical stresses associated with probe insertion and positioning operations. The connector system may be designed to support high-channel-count probe configurations while maintaining electrical signal integrity.

Probe 1478 may be configured with multiple recording sites that provide comprehensive measurement capabilities for organoid analysis. The probe configuration may be optimized for compatibility with the connector system while maintaining the electrical and mechanical performance required for organoid probing operations. The probe design may accommodate different organoid sizes and measurement requirements while maintaining compatibility with automated positioning systems.

As shown in FIG. 17, the alternative connector system may enable probe board 1471 to be easily replaced while maintaining reliable electrical connections with motherboard components. The connector configuration may provide positive mechanical engagement that prevents accidental disconnection during probe insertion operations while enabling easy removal for probe replacement procedures. The connector system may support high-frequency electrical signals while maintaining mechanical stability during dynamic probe positioning operations

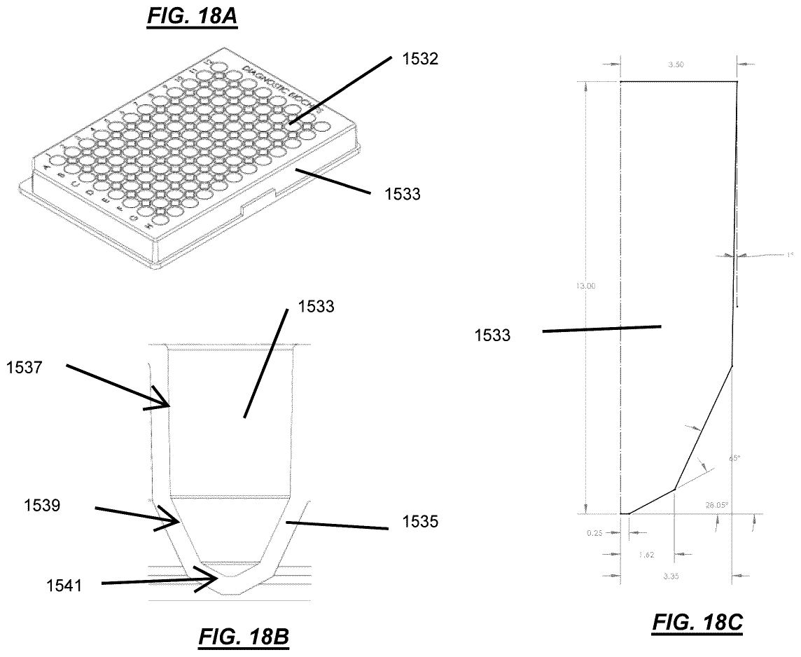

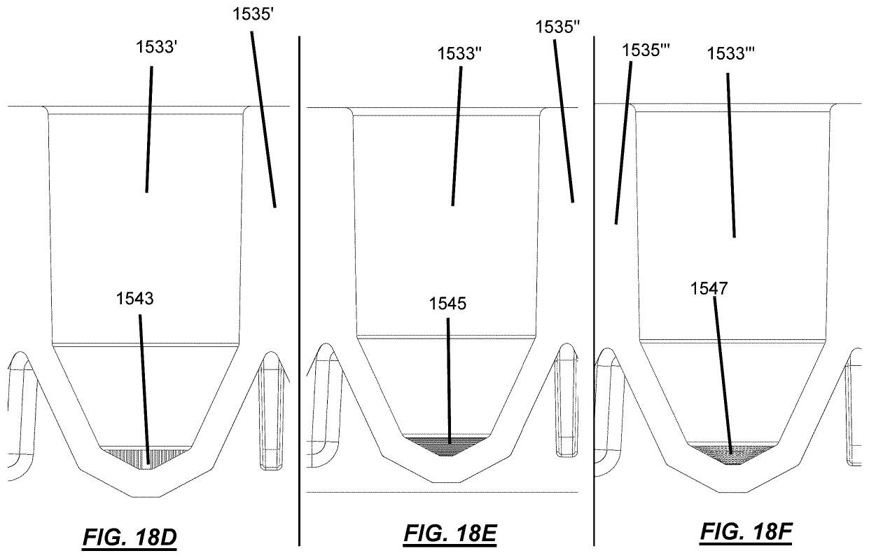

Referring to FIG. 18A-18C, a microwell plate 1532 may be configured with specialized well geometries that optimize organoid positioning and containment during measurement operations. Microwell plate 1532 may include wells 1533 having a first portion 1537, a second portion 1539, and a third portion 1541 with different shapes that provide systematic organoid guidance and positioning. Each well 1533 may include a wall 1535 that defines the well geometry and provides containment for organoids during measurement procedures.

With continued reference to FIG. 18A-18C, the first portion 1537 may have a different shape than the second portion 1539, and the second portion 1539 may have a different shape than the third portion 1541. The progressive shape changes may provide systematic guidance that directs organoids toward optimal positioning within wells 1533 while providing increasing containment as organoids descend into the wells. The multi-portion well design may optimize both organoid positioning and probe access during measurement operations.

The well geometry may be configured such that probes are configured to extend at least into the second portion 1539 of wells 1533 during measurement operations. The probe extension into the second portion may ensure optimal probe positioning relative to organoids while maintaining compatibility with the well geometry and organoid containment characteristics. The probe positioning may be optimized to provide maximum measurement quality while preventing damage to organoid tissue during insertion procedures.

As shown in FIG. 18A-18C, the three-portion well design may provide optimized organoid positioning through systematic guidance and containment. The first portion 1537 may provide initial guidance for organoid placement, the second portion 1539 may provide intermediate containment and probe access, and the third portion 1541 may provide final positioning and support for organoids during measurement operations. The progressive well geometry may accommodate organoids of varying sizes while maintaining consistent positioning characteristics.

The well profile may have a 2-step taper which terminates at a flat circle. The 2-step taper serves to 1) guide the organoid to the center of the well; 2) contain the organoid in the center during insertion (prevent slipping); and 3) minimize surface contact with the organoid which can prevent nutrients from reaching cells. The bottom surface may be polished for optical transparency and organoid visualization.

Referring to FIG. 18D, FIG. 18E, and FIG. 18F, alternative well configurations may include surface features that enhance organoid positioning and containment capabilities. Shown in FIG. 18D, a well 1533′ may include a wall 1535′ having a feature 1543 that provides enhanced fixation or reduced contact characteristics. Shown in FIG. 18E, a well 1533′ may include a wall 1535′ having a feature 1545 that provides alternative surface characteristics for organoid interaction. Shown in FIG. 18F, a well 1533′″ may include a wall 1535′″ having a feature 1547 that provides additional surface modification options.

Feature 1543 may be configured as a channel, protrusion, or recess that modifies the interaction between organoid tissue and well surfaces. The surface features may increase fixation of organoids within wells by providing mechanical engagement points that prevent organoid displacement during probe insertion operations.

Feature 1545 may be configured to reduce contact between organoids and well surfaces, providing fluid channels that maintain nutrient access while supporting organoid positioning. The reduced contact configuration may optimize organoid health during extended measurement periods by ensuring adequate media exchange around organoid tissue. Feature 1547 may provide alternative surface modification options that balance fixation requirements with nutrient access needs based on specific experimental protocols.

As shown in FIG. 18D, FIG. 18E, and FIG. 18F, the surface features may be configured as channels that provide fluid pathways along well surfaces, enabling media circulation around organoids while maintaining positioning stability. The channel configuration may reduce surface contact area while providing mechanical guidance that maintains organoid positioning during measurement operations. The channels may be oriented to optimize fluid flow patterns that enhance nutrient delivery to organoid tissue.

Surface features may be configured as protrusions that provide discrete contact points with organoid tissue, reducing overall contact area while maintaining positioning stability. The protrusion configuration may enable precise control over organoid positioning while minimizing surface contact that could affect nutrient access or organoid health. The protrusions may be positioned to provide balanced support that prevents organoid displacement without excessive mechanical constraint.

With continued reference to FIG. 18D, FIG. 18E, and FIG. 18F, surface features may be configured as recesses that provide localized areas of reduced contact with organoid tissue. The recess configuration may create fluid reservoirs that enhance media exchange while maintaining overall well geometry that supports organoid positioning. The recesses may be positioned to optimize both mechanical support and nutrient access based on specific organoid characteristics and measurement requirements. Any or all of surface features 1543, 1545, 1547 may be added to all or portions of the inner walls of the wells to fix organoids in place or provide fluid channels that reduce surface contact. Any or all of surface features 1543, 1545, 1547 may be added to any or all of the wells of microwell plate 1532 (FIG. 18A). For example, some of the wells of microwell plate 1532 may include surface features 1543 and other wells may include surface features 1545 and 1547. Any combination of surface features 1543, 1545, 1547 of microwell plate 1532 may be contemplated.

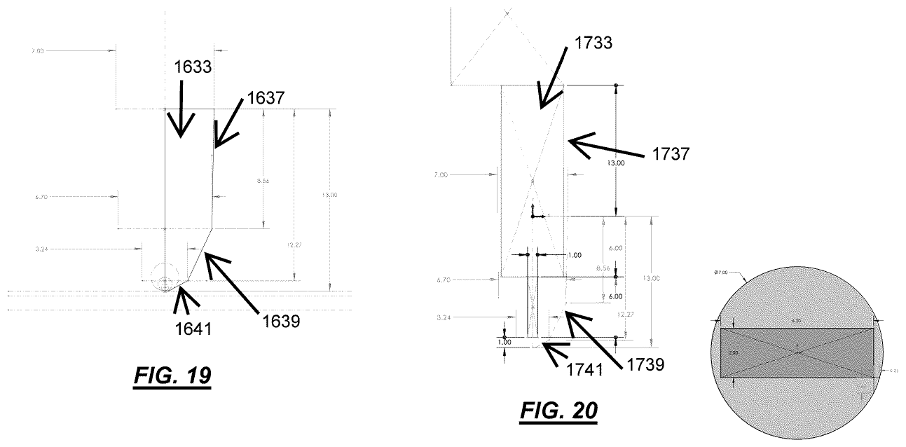

Referring to FIG. 19 and FIG. 20, alternative well geometries may provide different taper configurations that optimize organoid positioning for specific applications. A well 1633 may include a first portion 1637, a second portion 1639, and a third portion 1641 configured with specific angular relationships that optimize organoid guidance and containment. A well 1733 may include a first portion 1737, a second portion 1739, and a third portion 1741 configured with alternative angular relationships that provide different positioning characteristics.

With continued reference to FIG. 19 and FIG. 20, well 1633 may be configured such that a surface of second portion 1639 has a greater angle relative to vertical than first portion 1637, and wherein a surface of third portion 1641 has a greater angle relative to vertical than the outer surface of second portion 1639. The progressive angle increase may provide systematic guidance that directs organoids toward the center of well 1633 while providing increasing lateral support as organoids descend into the well. The angular configuration may optimize both organoid positioning and containment during measurement operations.

Well 1733 may be configured such that a surface of second portion 1739 has a smaller angle relative to vertical than a surface of first portion 1737 or a surface of third portion 1741. The reduced angle in second portion 1739 may provide a containment region that stabilizes organoid positioning while first portion 1737 and third portion 1741 provide guidance and support functions. The angular configuration may optimize organoid positioning by providing a stable containment region between guidance and support portions of the well.

As shown in FIG. 19 and FIG. 20, the angular relationships between different well portions may be optimized to provide specific organoid positioning characteristics based on organoid size, tissue properties, and measurement requirements. The progressive angle configuration of well 1633 may provide systematic guidance that ensures consistent organoid positioning across multiple loading operations. The variable angle configuration of well 1733 may provide enhanced containment capabilities that prevent organoid displacement during probe insertion procedures.

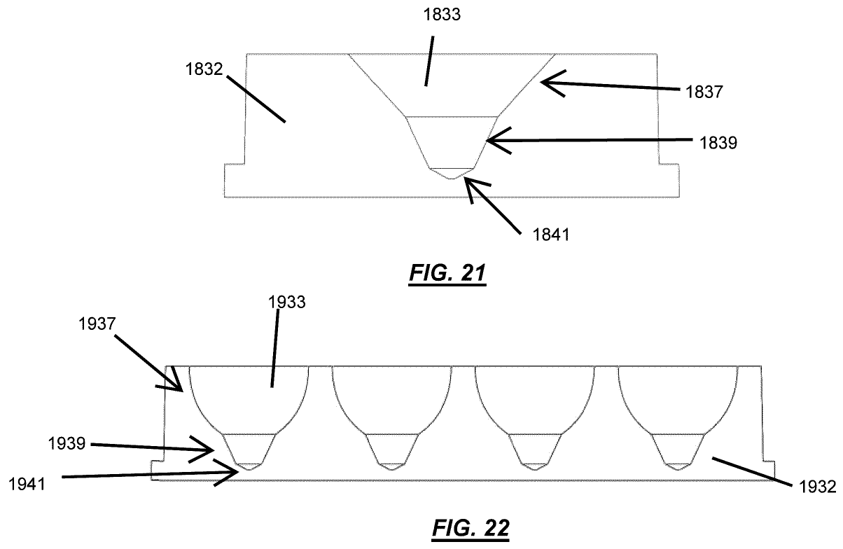

Referring to FIG. 21 and FIG. 22, additional well geometry variations may provide alternative organoid positioning capabilities through different combinations of curved and straight surface features. As shown in FIG. 21, a microwell plate 1832 may include wells 1833 having a first portion 1837, a second portion 1839, and a third portion 1841 configured with specific surface geometries. As shown in FIG. 22, a microwell plate 1932 may include wells 1933 having a first portion 1937, a second portion 1939, and a third portion 1941 configured with alternative surface geometries.

Referring to FIG. 21, wells 1833 may be configured such that a surface of first portion 1837 is curved, wherein a surface of second portion 1839 is straight, and wherein a surface of third portion 1841 is straight. The combination of curved and straight surfaces may provide optimized organoid guidance through the curved first portion while providing stable containment through the straight second and third portions. The surface of third portion 1841 may have a greater angle with respect to vertical than the surface of second portion 1839, providing progressive containment as organoids descend into the well.

Referring to FIG. 22, wells 1933 may be configured with alternative combinations of curved and straight surfaces that provide different organoid positioning characteristics. The surface geometry selection may be based on specific organoid characteristics, measurement requirements, and operational protocols. The curved surfaces may provide smooth guidance that minimizes mechanical stress on organoid tissue during loading operations, while straight surfaces may provide predictable containment characteristics that optimize probe access during measurement procedures.

As shown in FIG. 21 and FIG. 22, the well geometries may be optimized to provide specific organoid positioning characteristics while maintaining compatibility with probe insertion operations. The curved first portion may provide gentle guidance that accommodates variations in organoid placement during loading operations. The straight second and third portions may provide predictable containment that ensures consistent organoid positioning during probe insertion and measurement procedures.

The same well profile can be replicated on titer plates of varying sizes and well densities. Testing indicates that a 2-step taper may be a important for organoid alignment. Surface texture may be important for fixing the organoid in place. Plates may include single, 6, or 8 well configurations. The well opening may be increased to create a media ‘reservoir’. This reservoir may combat the need to replenish media loss due to evaporation during extended recording periods.

These wells may be produced via any manufacturing method, including, but not limited to, injection molding, 3D printing, CNC milling, etc. Other considerations may include insert molding a common ground wire that electrical connects and grounds all 96 wells simultaneously. Design inter-well, or individual well, fluid ports for drug delivery. Design a custom 96 well ‘insert’ that can modify any 96 well plate to match a desired profile. This insert could feasibly be reduced to any number of wells. A custom well insert that serves to compress/contain/align the organoid during insertion. The compression mechanism may to prevent ‘drift’ during recording. Extruded features located at the bottom of the well to aid in guiding the organoid to the center of the well. A titer plate lid that serves to provide a sort of self-contained environmental control and EMF shielding while having access ports for drug delivery and probe insertion.

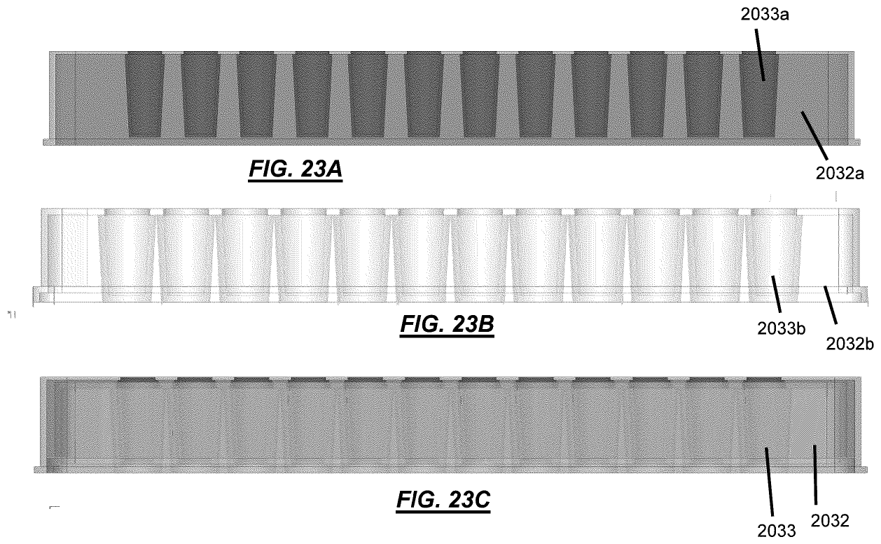

Referring to FIGS. 23A-23C, a modular microwell plate system may provide enhanced flexibility for customizing well geometries and surface characteristics. A microwell plate 2032 may include a plate insert 2032a and a plate shell 2032b that work together to provide customizable well configurations. Wells 2033 may be formed by well inserts 2033a positioned within well shells 2033b, enabling modification of well characteristics through insert replacement.

With continued reference to FIGS. 23A-23C, plate insert 2032a may be configured to be inserted into plate shell 2032b, creating a modular system that enables customization of well geometries without requiring complete plate replacement. The insert configuration may allow users to modify well characteristics by replacing plate insert 2032a while maintaining the same plate shell 2032b. The modular design may reduce costs while providing flexibility to accommodate different organoid types and measurement requirements. For example, the modular design may enable users to maintain a library of different well insert designs that can be rapidly deployed based on specific experimental requirements.

Well inserts 2033a may be configured with specific well geometries that optimize organoid positioning for particular applications. Different well insert designs may provide varying taper angles, surface features, or dimensional characteristics that accommodate different organoid sizes or measurement protocols. The well insert configuration may enable rapid customization of microwell plate 2032 by replacing well inserts 2033a without requiring modification of plate shell 2032b.

As shown in FIGS. 23A-23C, well shells 2033b may provide structural support and positioning for well inserts 2033a while maintaining compatibility with standard microwell plate dimensions. The well shell configuration may ensure proper alignment of well inserts 2033a while providing mechanical stability during organoid loading and probe insertion operations.

The modular microwell plate system may enable conversion of standard microwell plates into customized configurations through insertion of plate insert 2032a. The insert configuration may modify any standard microwell plate to match a desired well profile without requiring specialized plate manufacturing. The modular approach may provide cost-effective customization while maintaining compatibility with standard laboratory equipment and automation systems.

With continued reference to FIGS. 23A-23C, the insert design may be scaled to accommodate any number of wells within a microwell plate. The modular system may be configured for single-well applications or multi-well configurations based on specific throughput requirements. The scalable design may enable consistent well characteristics across different plate formats while maintaining the flexibility to customize individual well geometries through insert replacement.

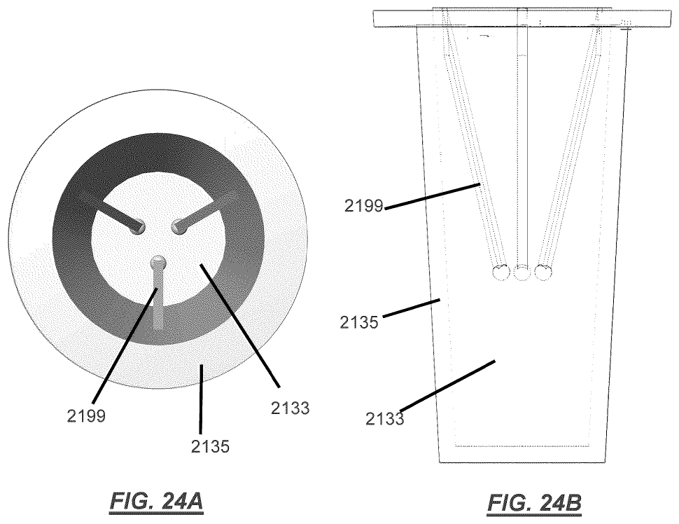

Referring to FIG. 24A and FIG. 24B, well guidance systems may provide enhanced organoid positioning capabilities through integrated guide structures. A well 2133 may include a wall 2135 and a guide 2199 that work together to direct organoids toward optimal positioning within the well. The guide system may provide mechanical guidance that ensures consistent organoid placement across multiple loading operations while maintaining compatibility with probe insertion procedures.

With continued reference to FIG. 24A and FIG. 24B, guide 2199 may extend from a top outer portion of well 2133 toward a bottom center portion of well 2133, providing directional guidance for organoid placement operations. The guide configuration may direct organoids toward the center of well 2133 during loading operations while providing mechanical support that maintains organoid positioning during probe insertion. Guide 2199 may extend at least halfway through a length of well 2133, wherein the length extends from a top of well 2133 to a bottom of well 2133, providing comprehensive guidance throughout the organoid descent path.

Well 2133 may include three guides 2199 positioned around the circumference of the well to provide balanced guidance for organoid positioning. The three-guide configuration may ensure consistent centering of organoids regardless of initial placement variations during loading operations. The guides may be evenly spaced around the well circumference to provide symmetric guidance that prevents organoid displacement toward well walls.

As shown in FIG. 24A and FIG. 24B, the guide configuration may provide both organoid guidance and probe guidance capabilities. The guides may direct organoids toward the center of well 2133 during loading operations while also providing mechanical guidance for probes during insertion procedures. The dual guidance function may optimize both organoid positioning and probe alignment, improving measurement consistency and reducing positioning errors during automated or manual probe insertion operations.

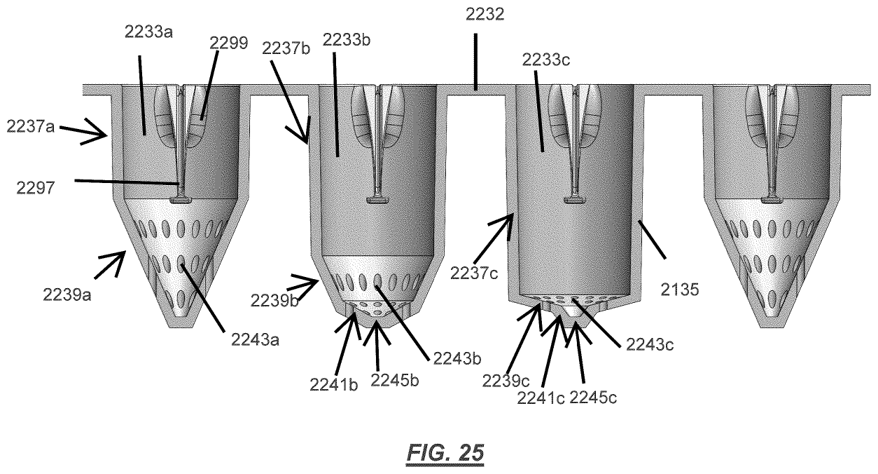

Referring to FIG. 25, alternative well configurations may incorporate funnel-shaped guide systems that provide enhanced organoid and probe positioning capabilities. A microwell plate 2232 may include wells having different guide configurations that optimize positioning for specific applications. Well 2233a may include a first portion 2237a, a second portion 2239a, and a feature 2243a configured to provide specific positioning characteristics. Well 2233b may include a first portion 2237b, a second portion 2239b, a third portion 2241b, a feature 2243b, and a bottom surface 2245b configured with alternative positioning features. Well 2233c may include a first portion 2237c, a second portion 2239c, a third portion 2241c, a feature 2243c, and a bottom surface 2245c configured with additional positioning options.

With continued reference to FIG. 25, a guide 2299 may include an upper portion and a lower portion 2297, wherein the upper portion is approximately funnel-shaped. The funnel-shaped upper portion may provide a wide acceptance area that accommodates variations in organoid or probe positioning while directing them toward the center of the well. The lower portion 2297 may be centered within the well and may extend approximately along a central longitudinal axis of the well, providing precise guidance for final positioning of organoids or probes.

The funnel-shaped guide configuration may provide systematic guidance that directs organoids or probes from a wide acceptance area toward a centered position within the well. The tapered funnel geometry may accommodate initial positioning variations while ensuring consistent final positioning that optimizes probe access and measurement quality. The guide system may be configured to work with both organoid placement operations and probe insertion procedures, providing comprehensive positioning support throughout the measurement workflow.

As shown in FIG. 25, the well configurations may include various combinations of tapered portions and guide features that optimize organoid positioning for specific applications. The first portions may provide initial guidance, the second portions may provide containment, and the third portions may provide final positioning support. The features may include surface modifications that enhance fixation or reduce contact between organoids and well surfaces, optimizing both positioning stability and organoid health during measurement operations.

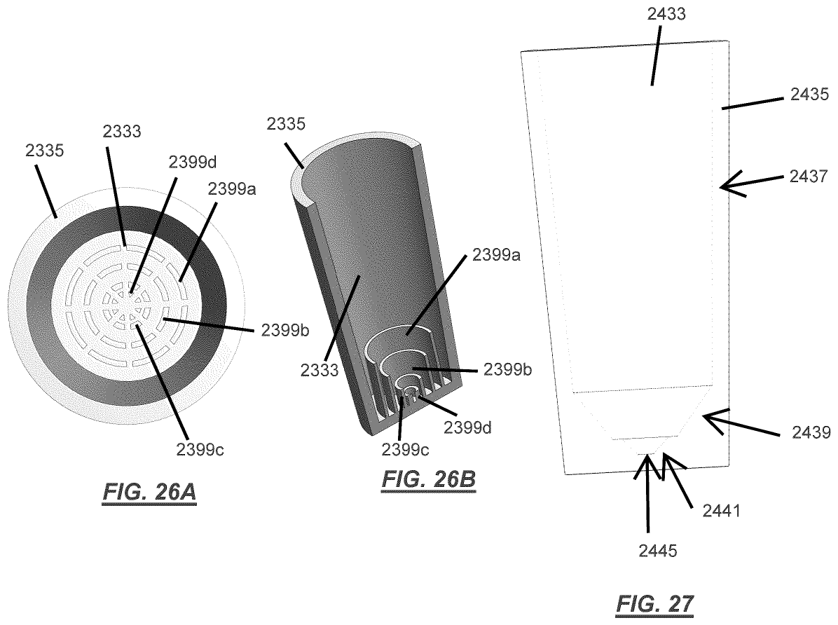

Referring to FIG. 26A, FIG. 26B, and FIG. 27, alternative well configurations may incorporate ring structures that provide enhanced organoid positioning and containment capabilities. A well 2333 may include a wall 2335 and multiple rings extending upward from a bottom surface of the well. The ring configuration may provide discrete support points that position organoids while minimizing surface contact that could affect nutrient access or organoid health.

With continued reference to FIG. 26A, FIG. 26B, and FIG. 27, well 2333 may include a first ring 2399a, a second ring 2399b, a third ring 2399c, and a fourth ring 2399d extending upward from a bottom surface of the well. The multiple ring configuration may provide progressive support that positions organoids at specific depths within the well based on organoid size. The rings may be concentric, providing symmetric support that maintains organoid centering during loading and measurement operations.

At least two of the rings may have different heights, providing stepped support that accommodates organoids of varying sizes. An outermost ring of the four rings may extend closer to a top of the well than an innermost ring of the four rings, creating a progressive support structure that guides organoids toward the center of the well while providing size-dependent positioning. The ring configuration may ensure that organoids of different sizes achieve optimal positioning for probe access during measurement operations.

As shown in FIG. 26A, FIG. 26B, and FIG. 27, the rings may be approximately cylindrical, providing consistent support characteristics around the organoid circumference. The cylindrical ring configuration may minimize contact area while providing stable positioning that prevents organoid displacement during probe insertion. The ring structures may be configured to provide fluid channels between rings that enhance media circulation around organoid tissue while maintaining positioning stability.

Well 2433 may be configured with a conical geometry having an 45° taper and an 0.5 mm-diameter base. Other geometries may be contemplated. The well may be approximately 9 mm deep, providing sufficient depth to accommodate organoids while maintaining appropriate positioning for probe access. Other depths may be contemplated. The conical geometry with slanted walls may provide lateral support that confines and stabilizes organoids during probe insertion and recording while minimizing physical contact to preserve media exchange and tissue health. Slanted walls may be beneficial to provide lateral support that confines and stabilizes the organoid during probe insertion and recording, while minimizing physical contact to preserve media exchange and tissue health.

With continued reference to FIG. 26A, FIG. 26B, and FIG. 27, the well may include a bottom surface 2437, a side wall 2439, an opening 2441, and a base 2445 that work together to provide optimized organoid containment. The bottom surface may be polished for optical transparency, enabling organoid visualization during measurement operations. The optical transparency may facilitate simultaneous optical and electrophysiological measurements, providing comprehensive organoid analysis capabilities.

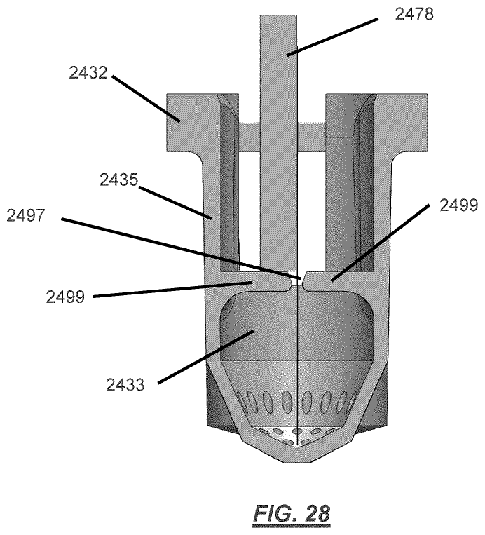

Referring to FIG. 28, well guidance systems may incorporate fin, rim, or lip structures that provide probe centering capabilities during insertion operations. A microwell plate 2432 may include a well 2433 having a wall 2435 and a fin 2499 that work together to guide probes toward the center of the well. A probe 2478 may be guided by fin 2499 through an opening 2497 defined by the fin structure, ensuring centered probe positioning during insertion operations.

With continued reference to FIG. 28, fin 2499 may define a central opening 2497 that guides probe 2478 into a center of well 2433. The fin configuration may provide mechanical guidance that corrects for minor probe positioning errors while directing probe 2478 toward the optimal insertion location within the well. The central opening may be sized to accommodate probe dimensions while providing sufficient guidance to ensure consistent probe centering across multiple insertion operations.

The fin, rim, or lip structure may be positioned at or near the top of well 2433 to provide early guidance for probe positioning during insertion procedures. The guidance structure may intercept probes before they contact organoid tissue, ensuring proper alignment before probe penetration begins. The early guidance may reduce the risk of probe misalignment that could damage organoid tissue or compromise measurement quality.

As shown in FIG. 28, the guidance structure may be configured to work in conjunction with well geometry to provide comprehensive positioning support for both organoids and probes. The fin may guide probes toward the center of the well while the well geometry positions organoids at the optimal location for probe access. The coordinated guidance system may optimize measurement consistency by ensuring proper alignment between probes and organoids during insertion operations.

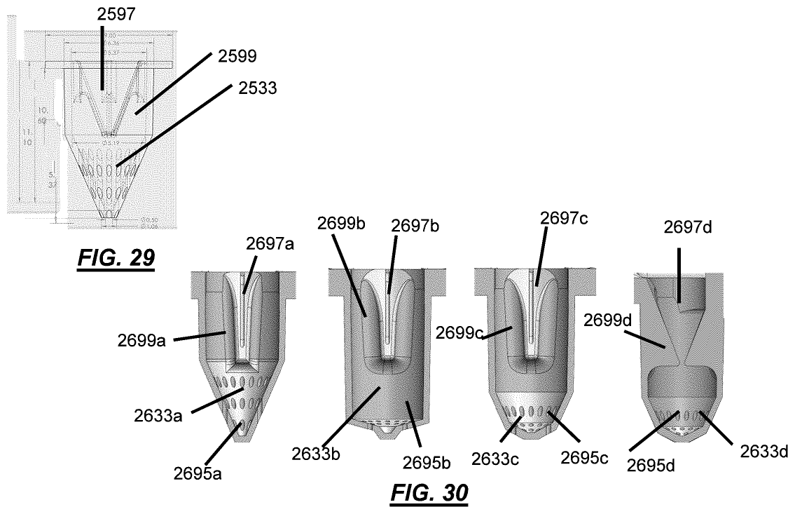

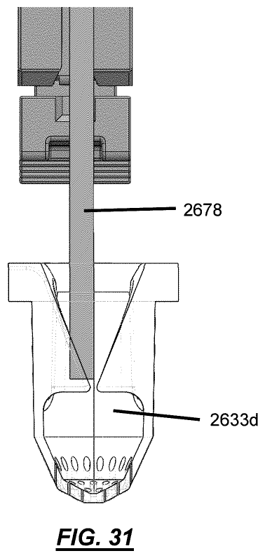

Referring to FIG. 29 and FIG. 30, tapered guide systems may provide enhanced probe positioning capabilities through funnel-shaped guidance structures. A well 2533 may include a guide 2599 having a funnel portion 2597 that provides systematic guidance for probe insertion. Multiple wells may be configured with different guide geometries that optimize positioning for specific applications. Well 2633a may include a guide 2699a having a funnel portion 2697a and a bottom portion 2695a. Well 2633b may include a guide 2699b having a funnel portion 2697b and a bottom portion 2695b. Well 2633c may include a guide 2699c having a funnel portion 2697c and a bottom portion 2695c. Well 2633d may include a guide 2699d having a funnel portion 2697d and a bottom portion 2695d.

With continued reference to FIG. 29 and FIG. 30, the tapered guides may define funnel-shaped portions at tops of microwells, providing wide acceptance areas that accommodate variations in probe positioning while directing probes toward centered positions within wells. The funnel-shaped portions may provide systematic guidance that corrects for positioning errors while maintaining smooth probe insertion trajectories that minimize mechanical stress on organoid tissue.

The tapered guides may be configured to guide probes into bottom portions of microwells such that probes are centered in the microwells. The guidance system may provide continuous directional support throughout the probe insertion process, ensuring consistent probe positioning from initial contact through final penetration depth. The centered positioning may optimize measurement quality by ensuring proper alignment between probe recording sites and organoid tissue.

As shown in FIG. 29 and FIG. 30, different guide configurations may provide varying levels of guidance support based on specific measurement requirements. The funnel portions may have different taper angles or depths that optimize guidance characteristics for different probe designs or insertion protocols. The bottom portions may provide final positioning support that ensures consistent probe centering at the organoid interface.

Referring to FIG. 31, the interaction between a probe head 2678 and well 2633d may illustrate the guidance capabilities provided by funnel-shaped guide systems. As probe head 2678 descends toward well 2633d, guide 2699d may provide systematic guidance that directs the probe toward the center of the well. The funnel portion 2697d may provide initial guidance that accommodates variations in probe head positioning, while bottom portion 2695d may provide final centering that ensures optimal probe alignment with the organoid.

With continued reference to FIG. 31, the tapered configuration of guide 2699d may direct the probe from probe head 2678 into bottom portion 2695d, ensuring that the probe achieves centered positioning within well 2633d during insertion operations. The funnel-shaped guidance provided by guide 2699d may accommodate variations in probe head 2678 positioning while maintaining consistent probe centering capabilities. The systematic guidance may enable probe head 2678 to achieve optimal probe positioning regardless of minor alignment variations during automated or manual positioning procedures.

The interaction between probe head 2678 and well 2633d may illustrate how the tapered guide system provides continuous guidance throughout the probe insertion process. As probe head 2678 descends, the probe may first contact the wide acceptance area of the funnel portion, then follow the tapered guidance pathway toward bottom portion 2695d where final centering occurs. The continuous guidance may ensure that the probe maintains optimal positioning throughout the insertion process, improving measurement accuracy and reducing the risk of probe misalignment during organoid probing operations.

As shown in FIG. 31, the funnel-shaped guide system may enable probe head 2678 to achieve consistent probe positioning across multiple measurement operations within well 2633d. The tapered guide may provide mechanical guidance that corrects for minor positioning errors while directing the probe toward the optimal insertion location within the well. The guide system may accommodate different probe head configurations and approach angles while maintaining consistent centering capabilities that optimize organoid measurement quality and reduce variability between different probe insertion procedures.

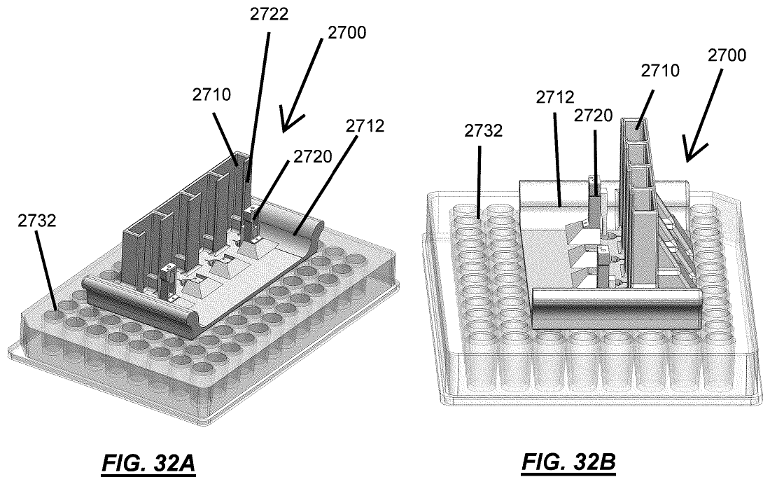

Referring to FIG. 31A and FIG. 32B, a system 2700 may be configured to provide simultaneous multi-well organoid probing capabilities through an integrated probe assembly and microwell plate configuration. System 2700 may include a probe assembly 2710, a lid portion 2712, probe heads 2720, drivers 2722, and a microwell plate 2732. The system 2700 may enable parallel measurement operations across multiple organoids while maintaining independent control over individual probe positioning and insertion parameters.

With continued reference to FIG. 31A and FIG. 32B, microwell plate 2732 may have a plurality of microwells, wherein each microwell of the plurality of microwells contains an organoid. The microwell plate 2732 may be configured to provide structural support and containment for multiple organoids during simultaneous measurement operations. Each microwell within microwell plate 2732 may be designed to accommodate individual organoids while providing access pathways for probe insertion from probe assembly 2710 positioned above microwell plate 2732.

Probe assembly 2710 may have a plurality of drivers 2722, wherein each driver is configured to drive a probe into a microwell of the plurality of microwells within microwell plate 2732. The probe assembly 2710 may provide coordinated control over multiple probe insertion operations while maintaining independent positioning capabilities for each individual probe. Each driver of drivers 2722 may be configured to control probe insertion depth, positioning, and timing for its respective microwell within microwell plate 2732.

As shown in FIG. 32A and FIG. 32B, probe assembly 2710 may form a lid for microwell plate 2732, creating an integrated measurement system that combines probe positioning capabilities with microwell containment functions. For example, probe assembly 2710 may include a custom designed microdrive may be built into a 96 well plate insert. The insert may be used with ‘chronic’ silicon probes and headstages, as described below. The lid configuration may enable probe assembly 2710 to be positioned directly over microwell plate 2732, providing optimal probe access to organoids within individual microwells while maintaining environmental control and containment during measurement operations. Lid portion 2712 may provide the structural framework that enables probe assembly 2710 to function as a lid while supporting the mechanical and electrical components required for multi-probe operations.

With continued reference to FIG. 32A and FIG. 32B, each of drivers 2722 may be independently controllable, enabling customized probe insertion parameters for individual microwells within microwell plate 2732. The independent control capability may allow each driver to accommodate variations in organoid size, position, or measurement requirements across different microwells while maintaining simultaneous operation capabilities. Each driver of drivers 2722 may be configured to provide precise positioning control that optimizes probe insertion for its respective organoid while coordinating with other drivers to maintain overall system performance.

Probe heads 2720 may be positioned within probe assembly 2710 and may be controlled by respective drivers 2722 to provide probe insertion capabilities for individual microwells within microwell plate 2732. Each probe head of probe heads 2720 may be configured to insert a probe into a specific microwell while maintaining independent positioning control through its associated driver. The probe heads 2720 may be arranged in a pattern that corresponds to the microwell arrangement within microwell plate 2732, enabling systematic coverage of all organoids during simultaneous measurement operations.

As shown in FIG. 32A and FIG. 32B, probe assembly 2710 may include a controller that coordinates operation of drivers 2722 while maintaining independent control capabilities for each individual driver. The controller may provide centralized command and control functions that enable synchronized probe insertion operations across multiple microwells while allowing customization of insertion parameters for individual organoids. The controller configuration may enable probe assembly 2710 to execute complex measurement protocols that require coordinated timing between different probe insertion operations while maintaining independent positioning control for each probe.

The integrated lid and probe assembly configuration may provide environmental control capabilities that maintain appropriate conditions for organoid viability during extended measurement operations. Probe assembly 2710 may function as a lid that seals microwell plate 2732 while providing controlled access for probe insertion operations. The lid configuration may maintain temperature, humidity, and atmospheric conditions within microwell plate 2732 while enabling simultaneous probe access to multiple organoids through controlled insertion mechanisms provided by drivers 2722.

With continued reference to FIG. 32A and FIG. 32B, the simultaneous multi-well probing capabilities of system 2700 may enable high-throughput organoid analysis while maintaining measurement quality and precision for individual organoids. Each driver of drivers 2722 may provide independent positioning control that accommodates variations in organoid characteristics while maintaining coordinated operation with other drivers within probe assembly 2710. The independent driver control may enable optimization of probe insertion parameters for each organoid while maintaining simultaneous measurement capabilities across the full array of microwells within microwell plate 2732.

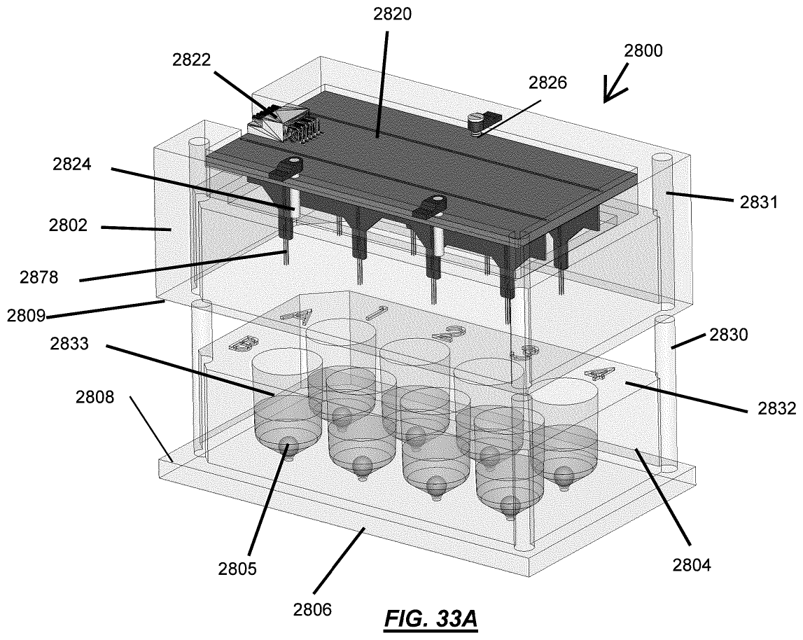

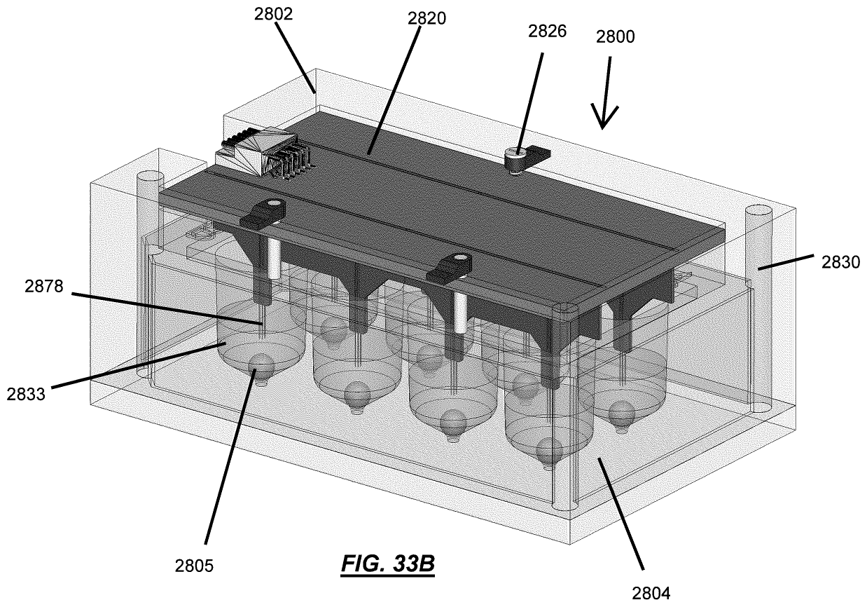

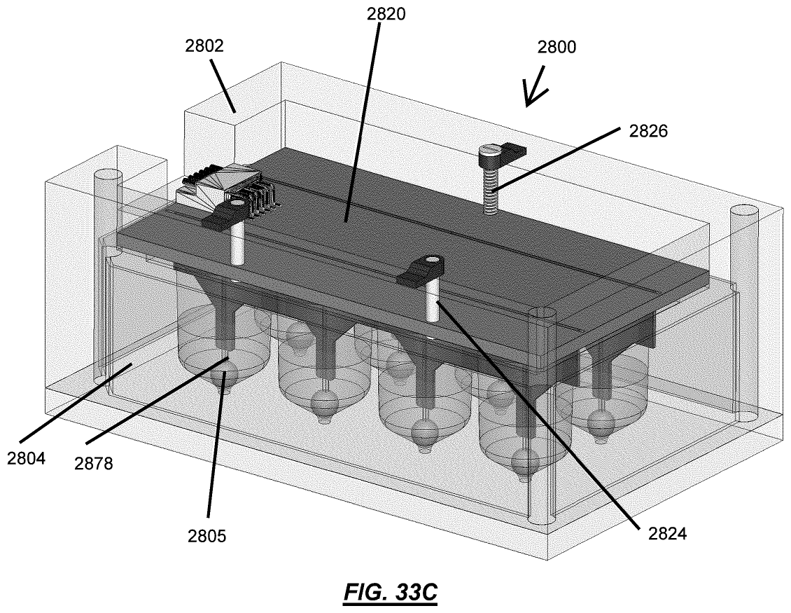

Referring to FIG. 33A, a system 2800 may be configured as a two-portion organoid probing system that provides adjustable probe positioning capabilities through a mechanically actuated probe plate mechanism. System 2800 may include a top portion 2802 and a bottom portion 2804 that work together to provide controlled probe insertion into organoids during measurement operations. An organoid 2805 may be positioned within system 2800 for electrophysiological analysis using probes that may be selectively positioned through mechanical actuation of internal components. For example, system 2800 may include a plate with include an integrated lid. The integrated lid may contains several probes connected to a motherboard. Alignment features on the lid and titerplate may ensure probe alignment over well center. An ultra-fine threaded screw may provide controlled z-actuation for probe insertion.

With continued reference to FIG. 33A, top portion 2802 may include a base 2806 having a top surface 2808 and a bottom surface 2809 that provide structural support for probe positioning mechanisms within system 2800. A probe plate 2820 may be positioned within top portion 2802 and may be configured to support multiple probes for organoid measurement operations. An electronic element 2822 may be positioned within top portion 2802 to provide signal processing and control functions for probes during organoid analysis procedures.

An alignment post 2824 may be positioned within top portion 2802 and may be configured to be received in a cavity of bottom portion 2804 to ensure proper alignment between top portion 2802 and bottom portion 2804 during system operation. A screw 2826 may be positioned within top portion 2802 to provide mechanical actuation capabilities that enable controlled positioning of probe plate 2820 relative to base 2806. The screw 2826 may provide precise positioning control that enables probe plate 2820 to be lowered or raised based on measurement requirements and operational protocols.

As shown in FIG. 33A, bottom portion 2804 may include a guide rail 2830 that provides mechanical guidance for interaction with top portion 2802 during system assembly and operation. A cavity 2831 may be defined within top portion 2802 and may be configured to receive guide rail 2830 when top portion 2802 is positioned on bottom portion 2804. A microwell plate 2832 may be positioned within bottom portion 2804 and may include a well 2833 configured to contain organoid 2805 during measurement operations.

A probe 2878 may extend from probe plate 2820 and may be configured to insert into organoid 2805 within well 2833 during measurement operations. Probe 2878 may be positioned such that the positioning of probe 2878 relative to organoid 2805 may be controlled through mechanical actuation of probe plate 2820 using screw 2826. The mechanical actuation system may enable system 2800 to operate in different configurations that provide varying levels of probe penetration into organoid 2805 based on measurement requirements.

With continued reference to FIG. 33A, system 2800 may be configured to operate in a first configuration where top portion 2802 is positioned on bottom portion 2804 and probes do not penetrate organoids within microwell plate 2832. In the first configuration, a housing of top portion 2802 may be fully lowered onto a housing of bottom portion 2804, creating a sealed measurement environment while maintaining separation between probes and organoids. Guide rail 2830 may be received fully within cavity 2831 of top portion 2802 in the first configuration, providing mechanical alignment and structural support between top portion 2802 and bottom portion 2804.

Referring to FIG. 33B, system 2800 may be shown in the first configuration where top portion 2802 is positioned on bottom portion 2804 without probe penetration into organoid 2805. Top portion 2802 may include probe plate 2820 positioned such that probe 2878 does not extend into well 2833 containing organoid 2805. Screw 2826 may be positioned to maintain probe plate 2820 at an elevated position relative to bottom surface 2809 of base 2806, preventing probe 2878 from contacting organoid 2805 during the first configuration.

With continued reference to FIG. 33B, guide rail 2830 may extend upward from bottom portion 2804 and may be received within cavity 2831 of top portion 2802, providing mechanical alignment between the two portions of system 2800. The guide rail 2830 configuration may ensure proper positioning of top portion 2802 relative to bottom portion 2804 while enabling controlled transition between different operational configurations. Organoid 2805 may be positioned within well 2833 and may be maintained in a stable environment during the first configuration while remaining accessible for probe insertion during subsequent configuration changes.

Referring to FIG. 33C, system 2800 may be shown in a second configuration where probe plate 2820 of top portion 2802 is lowered relative to a housing of top portion 2802, such that probes penetrate organoids within microwell plate 2832. The probe plate 2820 may be lowered by screw 2826, which may provide controlled mechanical actuation that positions probe 2878 for insertion into organoid 2805. Alignment post 2824 may be configured such that a portion of alignment post 2824 protrudes above probe plate 2820 in the second configuration, maintaining alignment between system components during probe insertion operations.

With continued reference to FIG. 33C, the alignment post 2824 may be configured as a post that provides mechanical registration between top portion 2802 and bottom portion 2804 during probe insertion operations. The post configuration may ensure consistent positioning of probe 2878 relative to organoid 2805 while accommodating the mechanical movement of probe plate 2820 during transition between operational configurations. Probe 2878 may extend from probe plate 2820 into organoid 2805 within well 2833, enabling electrophysiological measurements during the second configuration.

As shown in FIG. 33C, pressure may be applied to organoids to immobilize organoids during the second configuration, ensuring stable positioning during probe insertion and measurement operations. The pressure application may be achieved through the controlled lowering of probe plate 2820, which may compress organoid 2805 against well 2833 while maintaining probe access for measurement operations. The immobilization pressure may optimize measurement stability while preventing organoid displacement during probe insertion procedures.

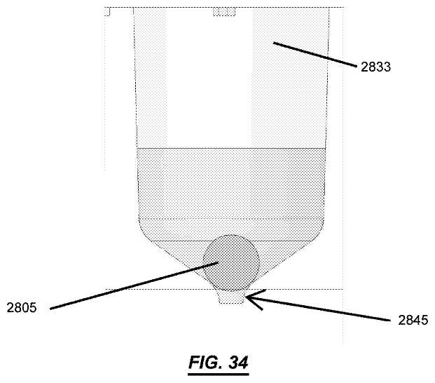

Referring to FIG. 34, well 2833 may include specialized geometric features that optimize organoid containment and probe access during measurement operations. A bottom portion 2845 of well 2833 may be narrower than organoid 2805 within the respective microwell, creating a containment configuration that positions organoid 2805 for optimal probe access while providing mechanical support during measurement operations. The narrowed bottom portion 2845 may prevent organoid 2805 from settling too deeply within well 2833 while maintaining stable positioning for probe insertion.

With continued reference to FIG. 33D, the geometric relationship between bottom portion 2845 and organoid 2805 may ensure that organoid 2805 remains accessible for probe insertion while being mechanically supported by the well structure. The narrowed configuration of bottom portion 2845 may create a platform effect that positions organoid 2805 at an optimal depth within well 2833 for probe access. The well geometry may accommodate organoids of varying sizes while maintaining consistent positioning characteristics that optimize measurement quality across different organoid samples.

System 2800 may include integrated temperature control capabilities through incorporation of heaters and other temperature controls within bottom portion 2804. The temperature control system may maintain optimal environmental conditions for organoid viability during extended measurement operations while providing precise thermal regulation that supports consistent measurement performance. The integrated heater configuration may enable system 2800 to maintain appropriate temperature conditions without requiring external heating systems or environmental chambers.

With continued reference to the system configurations shown in FIG. 33A, FIG. 33B, and FIG. 33C, top portion 2802 may form a lid of microwell plate 2832, creating an integrated measurement system that combines environmental control with probe positioning capabilities. The lid configuration may provide sealed environmental conditions that maintain organoid viability while enabling controlled probe access through mechanical actuation of probe plate 2820. Top portion 2802 or bottom portion 2804 may be at least partially transparent, enabling optical monitoring of organoid 2805 and probe 2878 positioning during measurement operations.

System 2800 may include perfusion features within top portion 2802 or bottom portion 2804 that provide controlled fluid delivery capabilities for media exchange, drug delivery, or other experimental protocols. The perfusion features may enable continuous or controlled delivery of compounds to organoid 2805 during measurement operations while maintaining the sealed environment provided by the lid configuration of top portion 2802. The integrated perfusion capabilities may support extended measurement protocols that require precise control over organoid environmental conditions.

Each probe of the plurality of probes within system 2800 may include a plurality of shanks that provide enhanced measurement capabilities for comprehensive organoid analysis. The multi-shank probe configuration may enable simultaneous measurement from multiple locations within organoid 2805 while maintaining the controlled positioning capabilities provided by the mechanical actuation system of probe plate 2820. The shank configuration may optimize spatial coverage of organoid electrical activity while maintaining compatibility with the two-configuration operational approach of system 2800.

System 2800 may comprise an automated tool loading mechanism that includes a pre-loaded cartridge of instrument tools for enhanced operational efficiency. The automated tool loading system may enable continuous operation across multiple measurement sessions while maintaining measurement quality through systematic tool replacement procedures. The cartridge-based approach may provide standardized tool handling that reduces manual intervention requirements while ensuring consistent tool performance across different measurement operations.

The automated tool exchange within system 2800 may be achieved by leveraging a friction release mechanism of an edge connector by pressing a tool into a tool retriever. The friction release approach may provide reliable tool removal and replacement capabilities without requiring complex mechanical actuation systems. The tool retriever configuration may enable systematic tool handling that maintains precise positioning while accommodating the consumable nature of probe components within system 2800.

Alternatively, an actuated gripper may translate to grab a tool and remove the tool from probe head components, deposit the used tool, grab a new tool, and insert the new tool into probe head components. The actuated gripper system may provide enhanced tool handling capabilities that accommodate different tool configurations while maintaining precise positioning control during tool exchange operations. The gripper-based approach may enable automated tool replacement that supports continuous operation of system 2800 across extended measurement protocols.

Automated loading capabilities within system 2800 may also include a shaker feature to preserve cell culture or organ samples as they wait to be loaded for testing. The shaker feature may provide controlled agitation that maintains organoid viability and prevents settling during storage periods between measurement operations. The preservation shaker may optimize organoid condition while supporting automated loading procedures that maintain measurement quality across multiple organoid samples within system 2800.

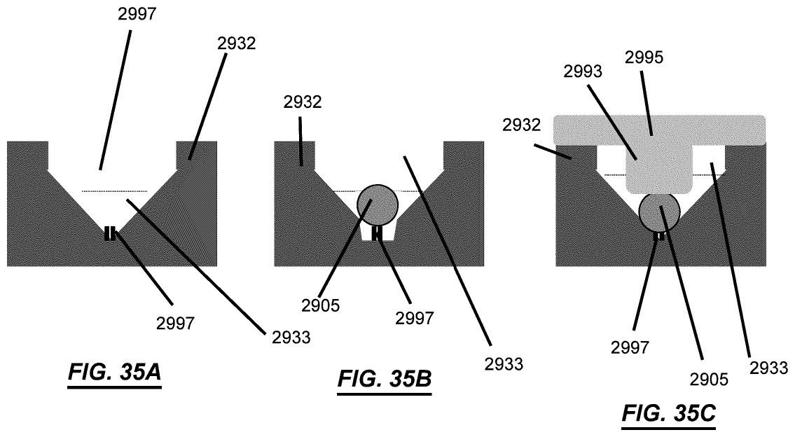

Referring to FIGS. 35A-35C, a microwell plate 2932 may be configured with integrated retention features that provide enhanced organoid positioning and containment capabilities during measurement operations. Probes may be integrated directly into the bottom of individual wells (through assembly, insert molding, or other means) of a titer plate. A lid may press and contain the organoid onto the probe shanks without damaging either the probes or the organoid. Alignment features can be incorporated to aid in guiding organoid to well center. Heaters and other temperature controls may be incorporated. Microwell plate 2932 may include a well 2933 configured to contain an organoid 2905 during electrophysiological analysis procedures. A retention feature 2999 may protrude from a bottom of well 2933 to provide mechanical engagement with organoid 2905 that secures organoid 2905 in place during probe insertion and measurement operations.

With continued reference to FIGS. 35A-35C, a cover 2995 may be configured to fit over microwell plate 2932 and may include a protrusion 2993 that provides compression capabilities for organoid positioning. Upon fitting cover 2995 to microwell plate 2932, protrusion 2993 may compress organoid 2905 onto retention feature 2999 such that retention feature 2999 engages with organoid 2905 to retain organoid 2905 in place. The compression mechanism may ensure stable organoid positioning during measurement operations while preventing organoid displacement that could affect measurement quality.

Retention feature 2999 may include at least one protrusion that extends upward from the bottom of well 2933 to provide mechanical engagement with organoid 2905. In some cases, retention feature 2999 may include at least two protrusions that provide enhanced organoid engagement through multiple contact points. The multiple protrusion configuration may distribute engagement forces across organoid 2905 while providing redundant retention capabilities that maintain organoid positioning even if individual protrusions experience reduced contact.

As shown in FIGS. 35A-35C, protrusion 2993 may be shaft-shaped, providing a defined geometric profile that enables controlled compression of organoid 2905 onto retention feature 2999. The shaft-shaped protrusion 2993 may have a smaller depth than a thickness of organoid 2905, ensuring that compression forces are applied without causing excessive deformation or damage to organoid 2905. The dimensional relationship between protrusion 2993 and organoid 2905 may optimize retention effectiveness while maintaining organoid viability during measurement operations.

When retention feature 2999 engages with organoid 2905, retention feature 2999 may puncture into organoid 2905 to provide secure mechanical attachment that prevents organoid displacement during probe insertion procedures. The puncturing engagement may create physical anchoring points within organoid 2905 that maintain positioning stability while allowing continued organoid function during electrophysiological measurements. The puncturing depth may be controlled to provide adequate retention without causing significant tissue damage that could affect measurement quality.

With continued reference to FIGS. 35A-35C, well 2933 may include a fill line 2997 indicating a fill point for media that maintains appropriate environmental conditions for organoid 2905 during measurement operations. Fill line 2997 may be positioned such that media covers organoid 2905 when well 2933 is filled to the indicated level. The media coverage may ensure adequate nutrient access and environmental conditions for organoid 2905 while maintaining appropriate fluid levels for measurement procedures. When filled to fill line 2997, the media may not be overly deep, preventing excessive fluid volume that could interfere with probe access or measurement operations.

In some cases, protrusion 2993 may be configured as a shank that provides both compression and measurement capabilities. The shank configuration may enable protrusion 2993 to function as a recording element while providing the compression forces needed to secure organoid 2905 onto retention feature 2999. The shank may include a recording site that enables electrophysiological measurements to be acquired through protrusion 2993 while organoid 2905 is compressed and retained in position by the engagement between protrusion 2993 and retention feature 2999.

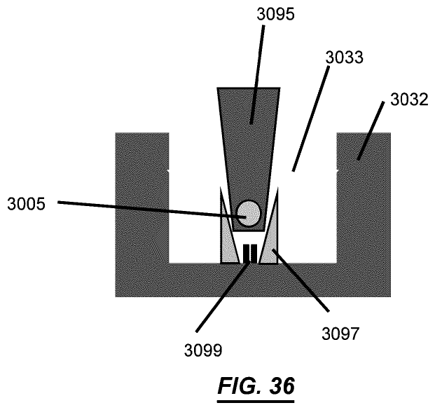

Referring to FIG. 36, a microwell plate 3032 may be configured with enhanced organoid positioning capabilities that combine retention features with guidance systems for improved organoid placement accuracy. Microwell plate 3032 may include a well 3033 configured to contain an organoid 3005 during measurement operations. A retention feature 3099 may protrude from a bottom of well 3033 to provide mechanical engagement with organoid 3005 that secures organoid 3005 in position during measurement procedures.

With continued reference to FIG. 36, a guide 3097 may protrude from the bottom of well 3033 to provide directional guidance for organoid placement operations. A pipette 3095 may be used for positioning organoid 3005 within well 3033, with guide 3097 configured to guide pipette 3095 to position organoid 3005 on retention feature 3099. The guide system may enable precise organoid placement that ensures optimal engagement between organoid 3005 and retention feature 3099 while reducing placement errors that could affect measurement quality.

Guide 3097 may extend upward from the bottom of well 3033, creating a three-dimensional guidance structure that directs pipette 3095 toward retention feature 3099 during organoid placement operations. Guide 3097 may have a wedge shape that provides convergent guidance pathways for pipette 3095 positioning. In some cases, microwell plate 3032 may comprise two guides having wedge shapes that work together to provide comprehensive organoid placement guidance.

As shown in FIG. 36 the two guides may form a funnel shape that creates a wide acceptance area for pipette 3095 while directing organoid 3005 toward retention feature 3099. The two guides may be positioned on either side of retention feature 3099, creating a balanced guidance system that centers organoid 3005 over retention feature 3099 during placement operations. The guides may taper toward a center of well 3033 moving from top to bottom, providing systematic guidance that ensures consistent organoid positioning across multiple placement procedures.

The wedge-shaped configuration of guide 3097 may accommodate pipette 3095 approach angles while providing directional guidance that positions organoid 3005 optimally relative to retention feature 3099. The funnel-shaped guidance created by multiple guides may enable reliable organoid placement even when pipette 3095 positioning varies between different placement operations. The tapered guidance may ensure that organoid 3005 settles into proper engagement with retention feature 3099 regardless of initial placement variations, improving measurement consistency across multiple organoid samples within microwell plate 3032.

Referring to FIG. 42A and FIG. 42B, a microwell plate 4032 may be configured with a well geometry that provides organoid positioning capabilities through a multi-portion well structure. Microwell plate 4032 may include a wall 4035 that defines the well boundary and provides containment for organoids during measurement operations. The well may include a first portion 4037 having a curved or bowl-shaped geometry that tapers downward toward a center of the well. A second portion 4041 may be positioned at a bottom center of the well, where the tapered walls of first portion 4037 converge to form a narrowed region.

The well geometry may transition from a wider opening at a top of the well through first portion 4037 to a narrower second portion 4041 at the bottom. The tapered configuration of first portion 4037 may guide organoids toward the center of the well.

With continued reference to FIG. 42A and FIG. 42B, second portion 4041 may include surface features that assist with the placement and containment of an organoid within the well. The surface features may provide enhanced organoid engagement that maintains positioning stability during measurement operations. In some aspects, the surface features may include ridges that extend along surfaces of second portion 4041 to provide mechanical engagement points with organoid tissue. The ridges may be oriented radially, circumferentially, or in other patterns that optimize organoid contact and retention within second portion 4041.

In some cases, the surface features may include roughened surfaces that increase friction between organoid tissue and the well surface. The roughened surfaces may be created through texturing, etching, or other surface modification techniques that provide microscale or nanoscale surface irregularities. The roughened configuration may reduce organoid movement during probe insertion procedures while maintaining organoid viability through controlled surface contact.

The surface features of second portion 4041 may work in conjunction with the tapered geometry of first portion 4037 to provide comprehensive organoid positioning capabilities. First portion 4037 may guide organoids toward second portion 4041, where the surface features may secure organoids in position for measurement operations. The combination of guidance and retention features may optimize organoid positioning consistency across multiple loading and measurement procedures within microwell plate 4032.

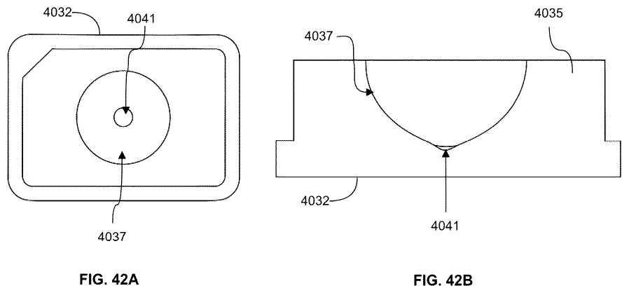

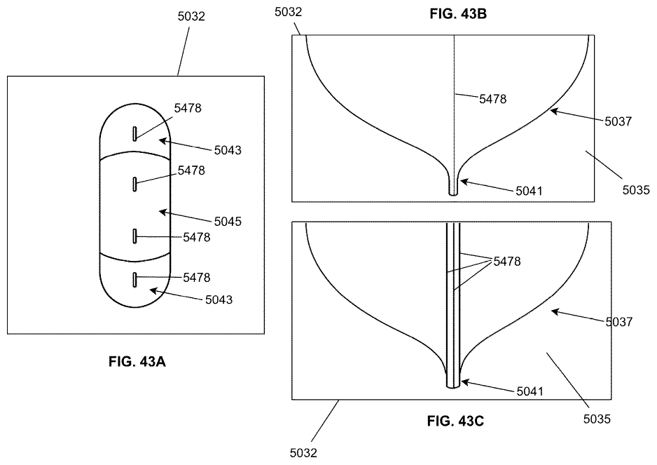

Referring to FIG. 43A, FIG. 43B, and FIG. 43C, a microwell plate 5032 may be configured with a well geometry that provides organoid positioning capabilities through a multi-portion well structure designed to accommodate organoids having a pancake or hockey puck shape. Microwell plate 5032 may include a wall 5035 that defines the well boundary and provides containment for organoids during measurement operations. The well may include a first portion 5037 having a curved or bowl-shaped geometry that tapers downward toward a center of the well. A second portion 5041 may be positioned at a bottom of the well, where the tapered walls of first portion 5037 converge to form a narrowed region configured to receive and position organoids for probing operations.

With continued reference to FIG. 43A, FIG. 43B, and FIG. 43C, second portion 5041 may include a third portion 5043 and a fourth portion 5045 that provide varying depths within the bottom region of the well. Fourth portion 5045 may be central to third portion 5043. Fourth portion 5045 may be deeper as compared to third portion 5043, creating a stepped or multi-level bottom configuration that accommodates organoid positioning. The elevated edges formed by third portion 5043 may support and position an organoid. In some configurations, the well may be produced with a uniform depth at the level of fourth portion 5045 without the elevated third portion 5043.

The well geometry may transition from a wider opening at a top of the well through first portion 5037 to a narrower second portion 5041 at the bottom. The tapered configuration of first portion 5037 may guide organoids toward the center of the well. Second portion 5041 may have steeper sides as compared to first portion 5037, providing enhanced lateral containment that confines and stabilizes organoids during probe insertion and measurement operations.

As shown in FIG. 43A, FIG. 43B, and FIG. 43C, one or more probes 5478 may extend into the well for electrophysiological measurements. Multiple probes 5478 may be positioned within the well, with some probes disposed within third portion 5043 and other probes disposed within fourth portion 5045. The probes may be oriented in various configurations to optimize measurement coverage across the organoid. During use, an organoid may fall to second portion 5041 and may be disposed within third portion 5043 and fourth portion 5045 of the well. The shape of second portion 5041 may allow for the organoid to be centered and positioned for accurate measurements and repeatability across multiple measurement sessions.

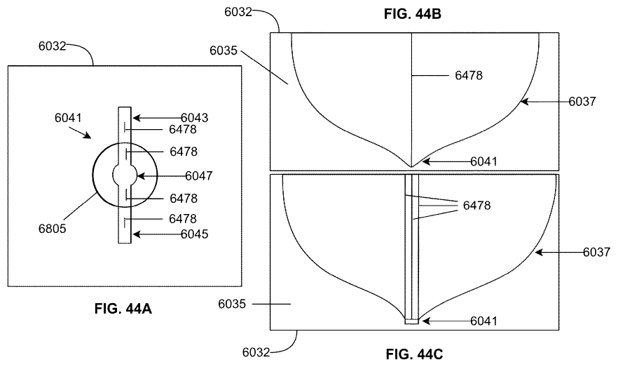

Referring to FIG. 44A, FIG. 44B, and FIG. 44C, a microwell plate 6032 may be configured with a well geometry that provides organoid positioning capabilities through a multi-portion well structure designed to accommodate organoids using an elongated channel configuration. Microwell plate 6032 may include a wall 6035 that defines the well boundary and provides containment for organoids during measurement operations. The well may include a first portion 6037 having a curved or bowl-shaped geometry that tapers downward toward a center of the well. A second portion 6041 may be positioned at a bottom of the well, where the tapered walls of first portion 6037 converge to form a narrowed region configured to receive and position organoids for probing operations.

With continued reference to FIG. 44A, FIG. 44B, and FIG. 44C, second portion 6041 may include an elongated channel having a first channel portion 6043, a second channel portion 6045, and a center portion 6047 disposed between first channel portion 6043 and second channel portion 6045. Center portion 6047 may be wider as compared to first channel portion 6043 and second channel portion 6045, creating a centering feature that assists with positioning an organoid 6805. The wider center portion 6047 may function as a pedestal-like structure that supports and centers organoid 6805 within the elongated channel configuration.

The well geometry may transition from a wider opening at a top of the well through first portion 6037 to a narrower second portion 6041 at the bottom. The tapered configuration of first portion 6037 may guide organoids toward the center of the well. The elongated channel of second portion 6041 may provide lateral containment that confines and stabilizes organoids during probe insertion and measurement operations.

As shown in FIG. 44A, FIG. 44B, and FIG. 44C, one or more probes 6478 may extend into the well for electrophysiological measurements. Multiple probes 6478 may be positioned within the well, extending through the elongated channel configuration. The probes may be oriented in various configurations to optimize measurement coverage across organoid 6805. During use, organoid 6805 may settle into second portion 6041 and may be disposed across center portion 6047 and at least a portion of each of first channel portion 6043 and second channel portion 6045. The shape of second portion 6041 and the centering feature provided by center portion 6047 may allow for organoid 6805 to be centered and positioned for accurate measurements and repeatability across multiple measurement sessions.

The well configurations described herein may be combined in various ways to provide customized organoid positioning and containment capabilities. For example, any of the wells described herein may include surface features such as ridges, roughened surfaces, channels, protrusions, or recesses to assist with positioning organoids and minimizing contact surface area between organoid tissue and well surfaces. The multi-portion well structures, tapered geometries, elongated channel configurations, retention features, and guide systems may be used in any combination based on specific organoid characteristics, measurement requirements, or experimental protocols. In some aspects, wells having stepped or multi-level bottom configurations may incorporate surface features within any portion of the well to enhance organoid engagement while maintaining nutrient access. The centering features, funnel-shaped guidance structures, and ring configurations described herein may be combined with any of the well geometries to provide comprehensive organoid positioning capabilities tailored to particular applications.

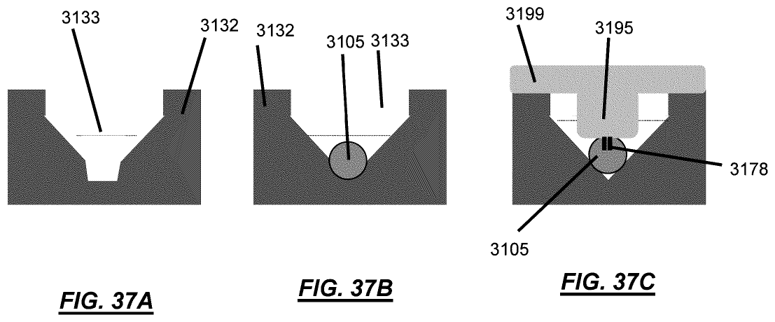

Referring to FIGS. 37A-37C, a microwell plate 3132 may be configured with an integrated lid system that provides enhanced organoid positioning and probe access capabilities through a cover-based probe delivery mechanism. Microwell plate 3132 may include a well 3133 configured to contain an organoid 3105 during electrophysiological measurement operations. A retention feature 3199 may be positioned within well 3133 to provide mechanical engagement with organoid 3105 that secures organoid 3105 in position during probe insertion and measurement procedures.

With continued reference to FIGS. 37A-37C, a cover 3195 may be configured to fit over microwell plate 3132 and may include a protrusion 3193 that provides both compression and probe delivery capabilities for organoid analysis. A probe 3178 may extend downward from protrusion 3193, creating an integrated probe delivery system that combines organoid positioning with measurement capabilities. Upon fitting cover 3195 to microwell plate 3132, probe 3178 may extend downward into organoid 3105 within well 3133, enabling electrophysiological measurements while maintaining organoid positioning through the compression provided by protrusion 3193.

The protrusion 3193 may be configured to compress organoid 3105 onto retention feature 3199 when cover 3195 is fitted to microwell plate 3132, ensuring stable organoid positioning during probe insertion and measurement operations. The compression mechanism may create secure mechanical engagement between organoid 3105 and retention feature 3199 while enabling probe 3178 to penetrate organoid 3105 for electrophysiological analysis. The integrated compression and probe delivery system may optimize measurement stability while maintaining organoid viability during extended recording periods.

As shown in FIGS. 37A-37C, microwell plate 3132 may be transparent, enabling optical monitoring of organoid 3105 and probe 3178 positioning during measurement operations. The transparent configuration may facilitate visual confirmation of probe insertion progress and organoid positioning while maintaining the structural integrity and containment capabilities of microwell plate 3132. The optical transparency may enable simultaneous optical and electrophysiological measurements, providing comprehensive organoid analysis capabilities through the integrated lid and probe system.

The retention feature 3199 may provide mechanical anchoring points within well 3133 that work in conjunction with the compression provided by protrusion 3193 to maintain organoid 3105 positioning during probe insertion procedures. The retention feature 3199 may be configured to engage with organoid 3105 when compression is applied through protrusion 3193, creating a stable positioning system that prevents organoid displacement during measurement operations. The combination of retention feature 3199 and protrusion 3193 may optimize organoid positioning while enabling reliable probe access for electrophysiological measurements.

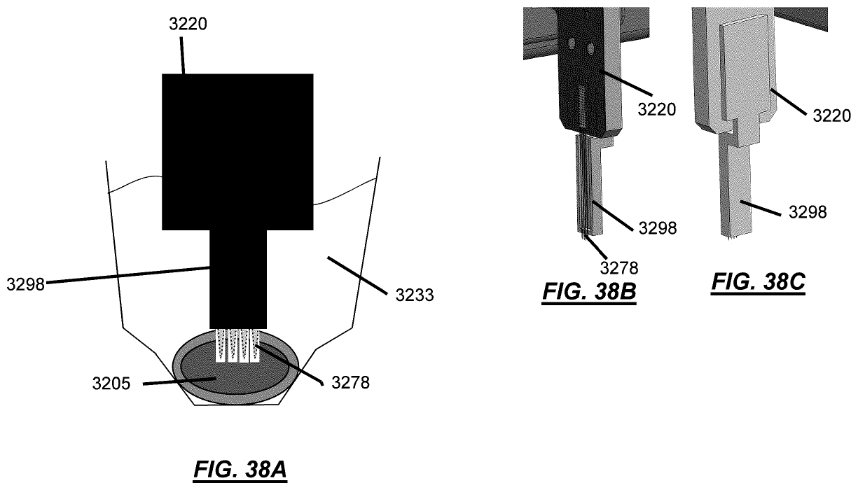

Referring to FIGS. 38A-38C, a probe head 3220 may be configured with a specialized cover system that provides organoid compression capabilities during probe insertion operations. Probe head 3220 may include a probe 3278 configured to insert into organoids for electrophysiological measurements. A cover 3298 may be positioned on probe head 3220 to cover at least part of a length of probe 3278, wherein cover 3298 may be configured to compress an organoid upon probing of the organoid using probe 3278.

With continued reference to FIGS. 38A-38C, an organoid 3205 may be positioned within a well 3233 for measurement operations using probe head 3220. Cover 3298 may be configured to compress organoid 3205 during probe insertion, providing mechanical stabilization that maintains organoid positioning while probe 3278 penetrates organoid 3205 for electrophysiological analysis. The compression provided by cover 3298 may prevent organoid displacement during probe insertion while maintaining organoid viability and measurement quality.

The probe 3278 may include a plurality of shanks that provide enhanced measurement capabilities for comprehensive organoid analysis. In some cases, probe head 3220 may include four shanks, enabling simultaneous measurement from multiple locations within organoid 3205 while maintaining the compression capabilities provided by cover 3298. The multi-shank configuration may optimize spatial coverage of organoid electrical activity while maintaining compatibility with the compression system provided by cover 3298.

As shown in FIGS. 38A-38C, cover 3298 may cover only one side of probe 3278, providing selective compression capabilities while maintaining probe access for organoid penetration. The single-side coverage may enable cover 3298 to provide compression forces against organoid 3205 while allowing probe 3278 to extend freely for insertion operations. Cover 3298 may be rectangular shaped, providing a defined geometric profile that enables controlled compression of organoid 3205 during measurement operations.

A tip of probe 3278 may extend below a distalmost end of cover 3298, ensuring that probe 3278 may penetrate organoid 3205 while cover 3298 provides compression support. The extended tip configuration may enable probe 3278 to achieve optimal insertion depth within organoid 3205 while maintaining the compression benefits provided by cover 3298. The dimensional relationship between probe 3278 and cover 3298 may optimize both measurement access and organoid positioning capabilities during electrophysiological analysis operations.

With continued reference to FIGS. 37A-37C and FIGS. 38A-38C, the lid-integrated probe systems may incorporate stimulation capabilities that enhance organoid analysis through controlled activation of neural tissue. An optical source may be utilized to stimulate organoids using optogenetics, wherein the optical source may be configured as a light-emitting diode, laser diode, or LED/LD coupled optical fiber positioned to deliver light to organoids during measurement operations. The optical stimulation system may enable selective activation or silencing of neurons within organoids while simultaneous electrophysiological measurements are acquired through probe 3178 or probe 3278. An optical source (LED, laser diode, or LED/LD coupled optical fiber close to where the endoscopic camera is) may be utilized to stimulate the culture cells or organoids. For example, the optical source may stimulate or silence the neurons using optogenetics.

Electrical wires may be used to electrically stimulate organoids during measurement operations, providing controlled electrical activation that complements the electrophysiological recording capabilities of the probe systems. The electrical stimulation wires may be positioned to deliver controlled electrical pulses to organoids while probe 3178 or probe 3278 acquire electrical responses from neural tissue. The combination of electrical stimulation and electrophysiological recording may enable comprehensive analysis of organoid neural function and response characteristics during measurement operations conducted through the lid-integrated probe systems. Electrical wires (close to where the GND/REF wires are) may be used to electrically stimulate the organoid or cell culture. Recording microelectrodes located on the probe shank may also be used to stimulate cell cultures/organoids (e.g., such as neurons).

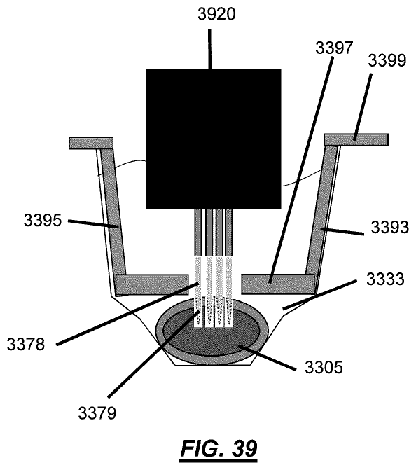

Referring to FIG. 39, a cover 3399 may be configured with a specialized opening system that enables probe access while providing organoid compression and positioning capabilities during measurement operations. Cover 3399 may include a protrusion 3395 that defines an opening 3333, wherein the opening 3333 enables a probe 3378 to extend through cover 3399 to probe an organoid 3305 within a microwell. A probe head 3920 may be positioned to deliver probe 3378 through opening 3333 for electrophysiological measurements of organoid 3305.

With continued reference to FIG. 39, protrusion 3395 may include a bottom wall 3397 and a side wall 3393 that work together to provide both structural support and organoid compression capabilities. The bottom wall 3397 may be configured to exert a downward pressure on organoid 3305, providing mechanical stabilization that maintains organoid positioning during probe insertion and measurement operations. Side wall 3393 may provide structural support for protrusion 3395 while defining the geometry of opening 3333 that accommodates probe 3378 during insertion procedures.

Protrusion 3395 may be hollow, creating an internal cavity that accommodates probe 3378 while maintaining the compression capabilities provided by bottom wall 3397. The hollow configuration may enable probe 3378 to extend through opening 3333 without mechanical interference while allowing bottom wall 3397 to contact organoid 3305 for positioning control. A top surface of bottom wall 3397 may be below a top surface of side wall 3393, creating a recessed configuration that optimizes organoid contact while maintaining structural integrity of protrusion 3395.

As shown in FIG. 39, probe 3378 may include recording sites 3379 positioned along the length of probe 3378 to provide comprehensive electrophysiological measurement capabilities. Recording sites 3379 may be configured as microelectrodes that acquire electrical signals from organoid 3305 during measurement operations.

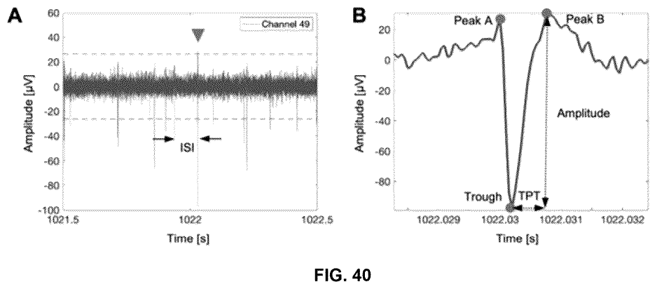

Referring to FIG. 40, an electrode of a probe may be marked active when a root mean square (RMS) extracellular voltage exceeds a predefined value, indicating presence of neurons and neuronal processes within an organoid. The following discussion applies to all probes described in this application. The probe may be a high-density microfabricated probe. In some embodiments, the probe may be made from silicon. The probe may be configured to be inserted into an organoid interior and acquire electrophysiological activity. The probe may include four 9 mm-long shanks, each carrying 16 microelectrodes. The shank may be 60 μm-wide (XZ plane) and 15 μm-thick (YZ plane). Recording sites may be distributed across a 750 μm lateral span (X axis) over the four shanks, and over 300 μm depth (Z axis) per shank. In some embodiments, the tip of a probe may be sharpened. The sharpened tips improve recording stability and allow reliable control of probe descent with predictable signal changes in response to incremental depth adjustments.

An RMS-based signal detection algorithm may identify onset and progression of neuronal activity within an organoid, and may halt insertion of a probe at a depth that maximizes active channel count among recording sites. A probe may be iteratively lowered at predetermined time increments, initially in approximately 50 micrometer steps, with pauses of 3 seconds between further lowering to acquire new measurements. Once a bottom-most electrode has an elevated RMS voltage above a threshold value (e.g., above 5.250±0.042 μV), indicating presence of neurons and neuronal processes (axons and dendrites) and their subthreshold signals, then the step size of further incremental lowering of the probe may be decreased to 20 micrometers, which may correspond to the vertical spacing between adjacent electrodes. An electrode may be marked as “active” when its updated RMS exceeds the predefined threshold. An algorithm may track progressive recruitment of the electrodes and halt insertion when a majority of the electrodes become active, indicating a desired (e.g., optimal) placement of the probe within the neuronal shell. When the probe is positioned in the organoid, the lowest electrodes may be active but not monitoring spikes, while the highest electrodes may not have yet penetrated the organoid (not be active). The electrodes in between the highest and the lowest organoids may measure spiking activities.

Impedance may be used to map an organoid, wherein a sensor measures impedance of signals using a test signal applied to the organoid. The impedance-based mapping may provide spatial characterization of the organoid that complements the electrophysiological measurements acquired through recording sites. A bandpass-filter may be applied to raw data collected by a probe, typically 300-6,000 Hz, to optimize signal quality for subsequent analysis procedures. In some embodiments, impedance may be used to map organoids. A sensor may measure the impedance of the signal and be used to map the organoid. For example, a test signal may be applied the organoid, and impedance may be measured using the same sensor.

Spikes may be detected based on a standard deviation from a mean, typically 5 SD, enabling identification of neural activity within an organoid. A 2 ms waveform starting 1 ms before a time of each spike may be extracted from each detected spike for detailed analysis. Features computed from waveforms may include inter-spike interval, amplitude, peak A, and trough-to-peak time, providing comprehensive characterization of neural activity patterns within an organoid. Any number of features may be computed from the waveform including but not limited to: (1) inter-spike interval (ISI); (2) amplitude; (3) peak A; and (4) trough-to-peak time (TPT). ISI may be the time duration between the spike and the most recent spike on the same channel. ISI may capture gross firing patterns at the multi-unit level, including bursting behavior. Amplitude may be the voltage difference between the trough of the spike waveform and the first ensuing peak. Positive spikes may be inverted prior to analysis. The amplitude feature may be signed to indicate negative (typically somatic) or positive (typically non-somatic) spikes. Peak A may be the (signed) voltage of the nearest peak that precedes the trough. A prominent Peak A may define a biphasic spike waveform, which may be associated with axonal potentials. TPT may be the time duration from the extremum (typically, the trough) to Peak B (e.g., the next peak). TPT may be a proxy for a width of the spike and useful for cell type classification.

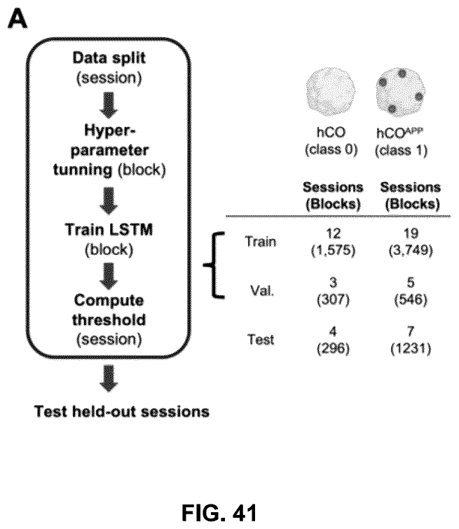

Referring to FIG. 41, post-processing artificial intelligence may utilize a long short-term memory (LSTM) classifier for automated phenotypic predictions from short electrophysiological recordings acquired through probes. The LSTM may be used to make binary predictions from brief recordings, ideally lasting only a few minutes, enabling rapid organoid analysis. The automated process may include probe insertion, spike recording, feature extraction, and classification. Classification may leverage a machine learning model configured to identify an organoid type, a mutation type, genetic mutations, pharmacological perturbations, or environmental toxicants based on waveform shape and spike timing characteristics acquired from organoids. The classifier may be trained using a dataset that may be split into a training dataset, a validation dataset, and a hold-out testing dataset. The classifier may output a binary classification, and precision, recall, and an F1-score may be calculated for each class. In some embodiments, the post-processing artificial intelligence may utilize a long short-term memory (LSTM) classifier. As noted above, the probing process may be automated. Use of the LTSM may allow the same probe to sample a large batch of organoids in rapid succession and produce accurate phenotypic predictions from short electrophysical recordings, ideally lasting only a few minutes. The LTSM may be used to make a binary prediction from brief recordings (e.g., distinguishing hCO from hCOAPP).

With continued reference to FIG. 41, a dataset may be split by session, reserving a percentage (e.g., 20%) of the sessions for testing. The remaining sessions may be split into blocks of consecutive spikes (e.g., 500 spikes) and used to train the LSTM model using a block-wise strategy. A validation split may be performed within the training set to guide optimization. During testing, predictions may be made at the block level. Once trained, a derived session-level classification may be derived by post hoc aggregation of the block-level model outputs. Specifically, each block classification score sᵢ ∈[0,1] may be transformed using a confidence-weighting function, w(sᵢ)=e^{c·|s}^{ᵢ}^{-0.5|}where c is a scaling constant (e.g., 4) that emphasizes high-confidence predictions near 0 or 1. The final session score ŷ may be defined as the weighted average of all blocks from that session: ŷ=(Σᵢ w(sᵢ)·sᵢ)/(Σᵢ w(sᵢ)). To determine a session-level classification threshold for ŷ, Youden's J statistic may be applied to the training and validation sessions, yielding an optimal cutoff. Model performance may be evaluated on held-out test sessions. An overview of model training and testing strategy is provided herein and depicted in the figure below. The dataset was first split by session, reserving a percentage (e.g., 20%) of the sessions for testing. The remaining sessions were split into blocks of consecutive spikes (e.g., 500 spikes) and used to train the LSTM model using a block-wise strategy. A validation split was performed within the training set to guide optimization. During testing, predictions were made at the block level.

Classification may be achieved using only data collected during a probe insertion phase, without requiring extended stationary recording, enabling real-time screening applications where the classifier operates concurrently with data acquisition, which is in turn synchronized with the instrument robotics. The model may accurately classify sessions using just a few minutes of insertion-phase activity. As the probe descended and encountered more active neurons, block accumulation accelerated and predictions converged rapidly. This insertion-only mode supports toward true real-time screening applications, where the classifier operates concurrently with data acquisition, which is in turn synchronized with the instrument robotics. Although a single binary classification task (hCO vs. hCOAPP) is provided as an example, the framework generalizes to other applications, including assessing genetic mutations, pharmacological perturbations, or environmental toxicants. For example, the EPA's ToxCast program has identified thousands of uncharacterized neurotoxicants that remain unscreened. Furthermore, the proposed platform may scale across brain regions, differentiation protocols, and even multi-center data, using transfer learning and model updating as new labeled datasets accumulate.

Any of the systems disclosed herein may have any of the following features, which apply to numerous of the above embodiments. The enclosure may include electromagnetic shielding. The enclosure may further include an amplifier. The amplifier may be mounted on an actuator. The actuator may be a motorized single-axis actuator. The system may be calibrated to achieve a predefined set of tolerances. For example, the system may be calibrated to maintain ≤100 μm lateral misalignment in the X/Y directions, 0.8 μm Z direction steps, and 15 μm repeatability in the Z direction. A well may be 9 mm deep, having conical geometry with a 45° taper and a 0.5 mm-diameter base. Slanted walls may be beneficial to provide lateral support that confines and stabilizes the organoid during probe insertion and recording, while minimizing physical contact to preserve media exchange and tissue health. Organoids may be of any dimension. In some embodiments, an organoid may have a diameter between 0.5-2 mm.

The tool may include a multi-probe head or a mutli-shank probe. For example, the tool may be comprised of one or more 64-channel probes. This allows multiple rows (or columns) to be scanned simultaneously, instead of one well at a time. Alternative instruments may feasibly consist of a tool with 1 to 1024 or more channels by scaling the ASIC and connector architecture of the probe head. Although rows and columns are described above, it will be appreciated that clusters or grids of wells/organoids may alternatively be simultaneously probed/scanned. Using multiple probes and probing multiple wells at the same time may increase efficiency.

One or more fluid port tips running along the PCB towards the probe for media exchange, drug delivery, and/or probe washing. In this configuration, the tool may have a quick connect to allow for fluid transport from the reservoirs housed in the enclosure. An endoscopic camera running along the PCB aiming towards the probe tips to serve as a visual aid for alignment and organoid insertion. Similarly, this configuration may include a quick connect to allow for data transmission of the camera to the instrument computer.

The following may be a representative workflow for the high-throughput system (e.g., of FIGS. 1-8). Organoids are loaded into a titer plate. The titer plate is loaded into the instrument. The instrument executes an organoid positioning procedure. The procedure may include gently ‘shaking’ the well to cause the organoids to fall to the well center. The instrument executes an alignment procedure.

The alignment procedure may include: a camera uses fiducials on the well plate and probe head to precisely locate the wells; a ‘start-up attachment’ is temporarily installed to precisely locate predefined positions on the well plate. The attachment may be replaced with an instrument tool following successful alignment.

With the alignment procedure complete, the instrument moves to the first well to penetrate a first organoid. Using closed-loop feedback the instrument locates the active cell layer of the first organoid and begins a recording. The instrument may repeat the procedure for the remaining wells following a pre-programmed or user-defined path. The recording data may be exported directly to the cloud for storage and/or post-processing. Such post-processing may include the utilization of artificial intelligence (AI) and/or spike sorting.

The following is an exemplary workflow for the low-throughput system (e.g., of FIGS. 9-11). An organoid is loaded into a titer plate. The titer plate is loaded into the instrument. The user fine-tunes XY location using manual stages and triggers instrument start. The instrument descends into the well. Using closed-loop feedback the instrument locates the active cell layer of the organoid and begins recording. The recording data is exported directly to the cloud for storage and post-processing (AI/Spike Sorting). In some aspects, vibration may be used during various steps above. For example, a titer or microwell plate may be vibrated. In other aspects, as a probe is removed from an organoid, the organoid may drop back into a well and accomplish vibrations.

The aspects described above may be combined in various ways that will be appreciated by those of skill in the art. For example, the microwell plates described above may be used with any of the probing systems described above. The above description is not limiting and the claims define the scope of protection.

We claim:

1. An organoid probing system comprising:
a probe head having a probe;
a first stage along which the probe head is movable in an X direction; and
a second stage configured to move a microwell plate in a Y direction, wherein the microwell plate has a microwell containing an organoid;
wherein the probe head is movable in a Z direction to insert the probe into the microwell or retract the probe away from the organoid in order to measure a property of the organoid.

2. The system of claim 1, wherein the microwell plate has a plurality of microwells each containing an organoid.

3. The system of claim 1, further comprising a washing station containing a fluid for washing the probe.

4. The system of claim 1, further comprising a camera.

5. The system of claim 4, wherein the microwell plate includes at least one fiducial, and wherein the camera is configured to use the at least one fiducial to align the probe head relative to the microwell.

6. The system of claim 1, further comprising:
a track coupled to the first stage; and
a slot defined within the track;
wherein the probe head is movable along the track through the slot.

7. The system of claim 1, further comprising a controller configured to control movement of the first stage and the second stage based on closed-loop feedback from electrophysiology data gathered from the probe.

8. The system of claim 1, further comprising at least one rotational stage to provide additional degrees of freedom for probe positioning.

9. The system of claim 1, further comprising an enclosure that provides environmental control and acts as a faraday cage to shield the probe head from electromagnetic interference.

10. The system of claim 1, wherein the probe head includes a motherboard and a probe board.

11. The system of claim 10 wherein the motherboard and the probe board are coupled to one another approximately orthogonally.

12. The system of claim 1, wherein a cover covers at least a portion of the probe in at least some configurations.

13. The system of claim 1, wherein the probe head includes a plurality of probes that are arranged linearly, such that a line extends through all of the probes.

14. The system of claim 13, wherein the microwell plate includes a plurality of microwells, wherein the probe head includes a cover that at least partially covers the probe, wherein the cover has a shape corresponding to a shape of a negative space between adjacent microwells of the microwell plate.

15. The system of claim 1, wherein the probe head includes a sensor configured to measure an impedance of the organoid.

16. A microwell plate for organoids comprising:
a plurality of wells, each well having:
a first portion;
a second portion; and
a third portion,
wherein the first portion has a different shape than the second portion, and wherein the second portion has a different shape than the third portion.

17. An organoid probing system comprising the plate of claim 16 and further comprising:
a probe head having a probe; and
a stage along which the probe head is movable;
wherein the probe head is configured to insert a probe of the probe head into a microwell of the plurality of microwells in order to measure a property of an organoid within the microwell, wherein the probe is configured to extend at least into the second portion of the microwell.

18. The plate of claim 17, wherein at least the third portion includes surface feature, wherein the surface feature increases fixation of an organoid within a well of the plurality of wells or reduces contact between an organoid within a well of the plurality of wells and a surface of the well.

19. An organoid probing system comprising:
a probe head comprising:
a motherboard; and
a probe board coupled to the motherboard and having a probe extending therefrom, wherein the probe board is configured as a consumable component removable from the motherboard;
a stage along which the probe head is movable; and
a microwell plate having a microwell containing an organoid;
wherein the probe head is configured to insert the probe into the microwell in order to measure a property of the organoid.

20. The organoid probing system of claim 19, further comprising:
a first stage along which the probe head is movable in an X direction; and
a second stage configured to move a microwell plate in a Y direction;
wherein the probe head is movable in a Z direction to insert the probe into the microwell or retract the probe away from the organoid in order to measure a property of the organoid.
